(19) United States
(12) Patent Application Publication
(10) Pub. No.: US 2022/0257494 A1
(43) Pub. Date: Aug. 18, 2022

(54) METHODS AND COMPOSITIONS FOR MICROFLILLNG THE SKIN WITH HYALURONIC ACID USING MICROCHANNEL TECHNOLOGY

(71) Applicant: AQUAVIT PHARMACEUTICALS, INC., New York, NY (US)

(72) Inventor: Sobin Chang, New York, NY (US)

(21) Appl. No.: 17/616,569

(22) PCT Filed: Jun. 4, 2020
(86) PCT No.: PCT/US20/36195

§ 371 Date: Dec. 3, 2021

Related U.S. Application Data
(60) Provisional application No. 62/856,740, filed on Jun. 4, 2019.

(51) Int. Cl. A61K 8/73; A61M 37/00; A61K 8/64

(52) U.S. Cl. CPC A61K 8/735; A61M 37/0015; A61K 8/64; A61M 2037/0061; A61K 2800/87; A61K 2800/91; A61K 2800/30; A61K 2800/84

(57) ABSTRACT

The present invention provides methods of administering a composition comprising hyaluronic acid to a subject's skin, comprising providing an effective amount of hyaluronic acid in solid form; constituting the hyaluronic acid in an aqueous solution; and administering the solution comprising hyaluronic acid to the subject's skin.

### CROSS-REFERENCE TO RELATED APPLICATIONS

This application claims the benefit of U.S. Provisional Appl. No. 62/856,740, filed Jun. 4, 2019, the contents of which are hereby incorporated by reference in their entirety.

### FIELD OF THE INVENTION

The field of the invention relates generally to the fields of pharmaceuticals, medical aesthetics, skin healthcare, dermatology, bioinstrumentation, bioengineering and biotechnology.

### BACKGROUND OF THE INVENTION

Hyaluronic acid (HA) is widely distributed in both prokaryotic and eukaryotic cells. In humans, HA is present in all tissues, especially in the skin. The biological functions of HA include hydration, lubrication of joints, filling capacity, and providing a framework for cells migration. HA also involves in tissue repair, wound healing and immune responses. In terms of cosmetic and aesthetic use, HA plays a role in hydration of the skin and reducing collagen deposition, which leads to reduced scarring.

Hyaluronic acids (HA) are commonly injected into a human body as a filler in medical aesthetics and orthopedics. In order to achieve results, they are usually cross-linked to sustain its form for a lengthy period of time until they dissolve/degrade into the skin. The cross-linking process turns HA into an injectable gel. Native hyaluronic acid has a relatively short half-life so various manufacturing techniques have been deployed to extend the length of the chain and stabilize the molecule for its use in medical applications. The introduction of protein-based cross-links, the introduction of free-radical scavenging molecules such as sorbitol, and minimal stabilization of the HA chains through chemical agents such as NASHA (non-animal stabilized hyaluronic acid) are all techniques that have been used. HA has been approved to treat osteoarthritis of the knee via intra-articular injection. Dry, scaly skin, such as that caused by atopic dermatitis, may be treated with skin lotion containing sodium hyaluronate as its active ingredient. HA has been used in various formulations to create artificial tears to treat dry eye.

HA is also a common ingredient in skin care products. HA is a polymer of disaccharides, themselves composed of D-glucuronic acid and N-acetyl-D-glucosamine, linked via alternating β-(1→4) and β-(1→3) glycosidic bonds. Hyaluronic acid can be 25,000 disaccharide repeats in length. Polymers of hyaluronic acid can range in size from 5,000 to 20,000,000 Da in vivo. The average molecular weight in human synovial fluid is 3-4 million Da or more, and hyaluronic acid purified from human umbilical cord is 3,140,000 Da. Hyaluronic acid also contains silicon, ranging between 350 μg/g to 1900 μg/g depending on location in the organism.

HA is energetically stable, in part because of the stereochemistry of its component disaccharides. Bulky groups on each sugar molecule are in sterically favored positions, whereas the smaller hydrogens assume the less-favorable axial positions.

HA is typically injected using either a classic sharp hypodermic needle or a micro-cannula. Complications include the severing of nerves and microvessels, pain, and bruising. In some cases, hyaluronic acid fillers result in a granulomatous foreign body reaction. HA is supplied in liquid form to practitioners and typically requires formulation with excipients to improve shelf life and stability, some of which may not be advantageous or desirable for certain applications.

What is needed are new and improved compositions and ways of administering HA into human skin.

### SUMMARY OF THE INVENTION

It is to be understood that both the foregoing general description of the embodiments and the following detailed description are exemplary, and thus do not restrict the scope of the embodiments.

In one aspect, the present invention provides compositions and methods for “microfill” where microchannel technology is used to deliver HA into the skin. Due to the viscosity of the currently available injectable HAs, and limitations and complications of classic needles or cannulas, it is extremely difficult to achieve the optimal results of microfill using current conventional approaches.

In some embodiments, methods and compositions herein utilize freeze, vacuum and/or oven dried HA crystals, or pure powder form of HA alone or combined with other agents in solid form, such as lyophilized agents. The compositions can be administered to subjects by “microfill” utilizing a microchannel delivery technology. The other agents, e.g., in lyophilized form, can include botulinum toxin (e.g., botulinum toxin of serotype A, B, C, D, E, F or G), vitamins, minerals, and/or PLLA (poly-L-lactic acid).

In some embodiments, the crystallization process allows for viscosity control of these therapeutic agents to transform into injectable compositions. HA's hydrophilic property allows the crystals to dissolve within seconds in aqueous solution prior to treatment. Furthermore, in some aspects, the compositions can be self-administered for skin care with an appropriate microchannel needle length.

These HA solids (in any forms, sizes, and shapes) can be provided in any combination of cross-linked and non-cross-linked for various purposes and/or personalization. Additional benefits can include increased durability, stability, improved skin health, and efficient manufacturing process and shelf life.

In some embodiments, the HA or combination of HA with one or more additional agents can be formulated and reconstituted as desired by the user, e.g., for intradermal delivery using a microneedle injection. In some embodiments, the compositions comprising HA can be administered to the skin in its pure form bypassing any processing steps in traditional HA injectable manufacturing processes.

In another aspect, the invention provides a method of administering a composition comprising hyaluronic acid to a subject's skin, comprising
i) providing an effective amount of hyaluronic acid in solid form;
ii) reconstituting the hyaluronic acid in an aqueous solution; and
iii) administering the solution comprising hyaluronic acid to the subject's skin.

In some embodiments, the hyaluronic acid in solid form is crystalline. In some embodiments, the hyaluronic acid in solid form is freeze dried or spray dried. In some embodiments, the hyaluronic acid in solid form is vacuum dried. In some embodiments, the hyaluronic acid in solid form is oven dried. In some embodiments, the hyaluronic acid is crosslinked. In some embodiments, the hyaluronic acid is non-crosslinked. In some embodiments, at least a portion of the hyaluronic acid is crosslinked. In some embodiments, at least a portion of the hyaluronic acid has different percentages of cross-linked and non-cross linked ratios.

In some embodiments, the hyaluronic acid in solid form is substantially pure. In some embodiments, the hyaluronic acid in solid form is in admixture with an effective amount of one or more additional bioactive agents. In some embodiments, the one or more bioactive agents in admixture is freeze dried, spray dried, vacuum dried and/or oven dried.

In some embodiments, the hyaluronic acid is reconstituted in a solution comprising an effective amount of one or more bioactive agents.

In some embodiments, the reconstituted solution of hyaluronic acid is combined with an effective amount of one or more bioactive agents in solid and/or liquid form. In some embodiments, the one or more bioactive agents in solid form is freeze dried, vacuum dried and/or oven dried. In some embodiments, the one or more bioactive agents in liquid form comprises an aqueous solution. In some embodiments, the one or more bioactive agents comprises a neuromodulator, vitamin, mineral, PLLA (poly-L-lactic acid) or any combination thereof. In some embodiments, the neuromodulator comprises botulinum toxin (e.g., botulinum toxin of serotype A, B, C, D, E, F or G).

In some embodiments, the hyaluronic acid in solid form is in the shape of flakes, a ball, a cube, or a star. The size of the shapes are not particularly limiting.

In some embodiments, the solution is administered to the subject's skin using a microneedle delivery device. In some embodiments, the microneedle delivery device comprises
i) a plurality of microneedles, wherein the microneedles are hollow or non-hollow, wherein one or multiple grooves are inset along an outer wall of the microneedles; and
ii) a reservoir capable of holding the solution to be delivered to a subject's skin,
   wherein the reservoir is attached to or contains a means to encourage flow of the solution into the tissue; wherein the solution is capable of being delivered into the skin by passing through the one or multiple grooves along the outer wall of the microneedle by a repeated motion of penetrating the microneedle delivery device into the skin of the subject.

In some embodiments, the hyaluronic acid in solid form is provided in a reservoir component of a microneedle device. In some embodiments, the hyaluronic acid is reconstituted in the reservoir component. In some embodiments, the reservoir container is made of glass. In some embodiments, the microneedles are non-hollow. In some embodiments, the means to encourage flow of the solution is gravity driven. In some embodiments, the means to encourage flow of the solution into the tissue is selected from the group consisting of a plunger, pump and suction mechanism. In some embodiments, the means to transport the flow of the solution into the tissue is a mechanical spring loaded pump system. In some embodiments, the microneedles have a single groove inset along the outer wall of the microneedle, wherein the single groove has a screw thread shape going clockwise or counterclockwise around the microneedle. In some embodiments, the microneedles are from 0.1 mm to about 1.0 mm in length and from 0.01 mm to about 0.2 mm in diameter. In some embodiments, the microneedles are composed of gold. In some embodiments, the plurality of microneedles comprises an array of microneedles in the shape of a circle. In some embodiments, the microneedles are made of 24-carat gold plated stainless steel and comprise an array of microneedles, e.g., an array of 20 microneedles.

In some embodiments, the one or more bioactive agents comprises a vitamin, a mineral, retinol, retinoic acid, a bleaching/whitening agent, stem cells, collagen, a neurotoxin, platelet-rich plasma, poly-L-lactic acid, an anesthetic r combinations thereof.

In another aspect, the invention provides a solid composition comprising an effective amount of hyaluronic acid in admixture with an effective amount of one or more bioactive agents. In some embodiments, the admixture is in the shape of flakes, a ball, a cube, or a star. In some embodiments, the composition is provided in a container. In some embodiments, the container is a reservoir component of a microneedle delivery device. In some embodiments, the admixture is substantially free of preservatives. In some embodiments, the composition is reconstituted into an injectable solution comprising the composition, wherein the composition has been reconstituted in aqueous solution, wherein the solution has a viscosity that enables efficient injection with a microneedle device.

In another aspect, the invention provides a microneedle device comprising a reservoir component, wherein the reservoir component comprises
i) an effective amount of hyaluronic acid in solid form; and
ii) optionally an effective amount of one or more bioactive agents in solid form.
   In some embodiments, the one or more bioactive agents is elected from the group consisting of one or more vitamins, one or more minerals, retinol, retinoic acid, a bleaching/whitening agent, collagen, a neuromodulator, poly-L-lactic acid, an anesthetic and combinations thereof. In some embodiments, the hyaluronic acid and one or more bioactive agents are not in admixture. In some embodiments, the hyaluronic acid and one or more bioactive agents are in admixture.

In some embodiments, the hyaluronic acid and/or bioactive agent is freeze dried. In some embodiments, the hyaluronic acid and/or bioactive agent is vacuum dried. In some embodiments, the hyaluronic acid and/or bioactive agent is oven dried. In some embodiments, the hyaluronic acid is crosslinked. In some embodiments, the hyaluronic acid is non-crosslinked. In some embodiments, at least a portion of the hyaluronic acid is crosslinked. In some embodiments, the hyaluronic acid in solid form is substantially pure. In some embodiments, the one or more bioactive agents comprises a neuromodulator. In some embodiments, the neuromodulator comprises botulinum toxin (e.g., botulinum toxin of serotype A, B, C, D, E, F or G).

In another aspect, the invention provides a kit comprising the compositions or microneedle devices as described herein, optionally comprising instructions for use and optionally further comprising one or more aqueous solutions for dissolving the hyaluronic acid and/or one or more bioactive agents. In some embodiments, the solution is a buffered solution. In some embodiments, the kit comprises one or more anesthetics. In some embodiments, the kit further comprises one or more containers comprising one or more bioactive agents in solid or liquid form. In some embodiments, the one or more bioactive agents is selected from the group consisting of one or more vitamins, one or more minerals, retinol, retinoic acid, a bleaching/whitening agent, collagen, a neuromodulator, poly-L-lactic acid, an anesthetic and combinations thereof.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1 is a view of a handheld microneedle injection apparatus. The syringe ejection volume is automatically controlled and dispenses into an interchangeable head containing one or several needles. The diagram shows the connection of corrugated connector and microneedle head. The rubber based connector is such that its flexibility will allow connections with small openings (1) and large ones (2) to fit and seal the microneedle head. The corrugated connector, also made of rubber (3), will further allow larger embodiments to connect to this system with the spring plate microneedle head (4).

FIG. 2 is an image of a screw on a microneedle head.

FIG. 3 is a schematic representation of a device in a syringe configuration. Alternative configurations include vial- and capsule-loaded configurations. The device holds a syringe (2) for automatic injection via one or more microneedles in the microneedle head. Ejection volume is controlled by an information processor (9). Other elements are noted: the motor or actuator (4) to control the piston (3), exchangeable and controllable needle head (1) and cam system and dial to adjust needle injection depth (5), and needle head ejector (10). Information is shown to the user in a display panel that may include a manual or touchscreen control panel (12) and data is stored in a storage unit (11) that may be removable. The needle head (1) may be controlled by an actuator (13).

FIG. 4 provides three additional views of a microneedle device. Microneedle components: (A) microneedles, (B) housing of the needles and (C) a reservoir.

FIG. 5 is a diagram showing the connection of corrugated connector and microneedle head. The rubber based connector is such that its flexibility will allow connections with small openings (1) and large ones (2) to fit and seal the microneedle head. The corrugated connector, also made of rubber (3), will further allow larger embodiments to connect to this system with the spring plate microneedle head (4).

FIG. 6 provides a depiction of the utility feature conferred by the circular or flat O-Rings. Said features enable enhanced liquid handling capabilities as evidenced by an airtight mechanism which facilitates the efficient and uniform delivery of treatment solutions to the skin. Said features are positioned at the interface of the cap and the reservoir channel so as to effectively prevent the leakage of treatment solution dosages. The RFID chip+O-ring depiction has been expanded. The cap/cover (1) will interface with the vial or container (5) containing a certain compound (6). The connection of both the cap/cover and the container may be sealed with a threaded opening (2). While pressure is applied vertically through the twisting motion of the thread, the rubber O-ring (3) seals the two interfaces (1) and (5) together. A ratchet mechanism (4) at the end will lock the cap in place. Embedded inside the rubber O-ring is a RFID chip (7) which material is shock, pH, temperature, and ozone resistant. The RFID chip will be stable enough under different environments to be able to effectively transmit data for applications such as data security, quality assurance/control, and logistics (8).

FIG. 7A-7B depict a utility feature conferred by the circular or flat O-Rings (FIG. 7A). Said features enable obvious and non-obvious advantages conferred by excellent weather and ozone resistance, temperature resistance (FIG. 7B) and the resistance to pH induced degradation of the butyl rubber or halogenated butyl rubber in comparison to other industrial rubbers and further addresses the stability of the material in the context of medical device utility, end user performance and pharmacological agent turbidity. Said features effectively enable enhanced material durability while preventing the leakage and inefficient delivery of treatment solution dosages with time.

FIG. 8 illustrates anti-unlock safety features of an O ring in a microneedle device.

FIG. 9 illustrates anti-unlock safety features of an O ring in a microneedle device.

FIG. 10 illustrates anti-unlock safety features of an O ring in a microneedle device.

FIG. 11 illustrates anti-unlock safety features of an O ring in a microneedle device.

FIG. 12 illustrates an exemplary microneedle drug delivery device.

FIG. 13 provides solid hyaluronic acid vacuum dried in a microneedle device.

FIG. 14 provides an exemplary microneedle device.

FIG. 15 provides internal assembly of parts of the device of FIG. 14.

FIG. 16 provides a view of the assembled internal parts of FIG. 14.

FIG. 17 provides a view of the assembled internal parts of FIG. 14.

FIG. 18 provides an external push assembly view of the device of FIG. 14.

FIG. 19 provides a view of the device of FIG. 14.

FIG. 20 provides a view of the device of FIG. 14.

FIG. 21 provides user operation of the device of FIG. 14.

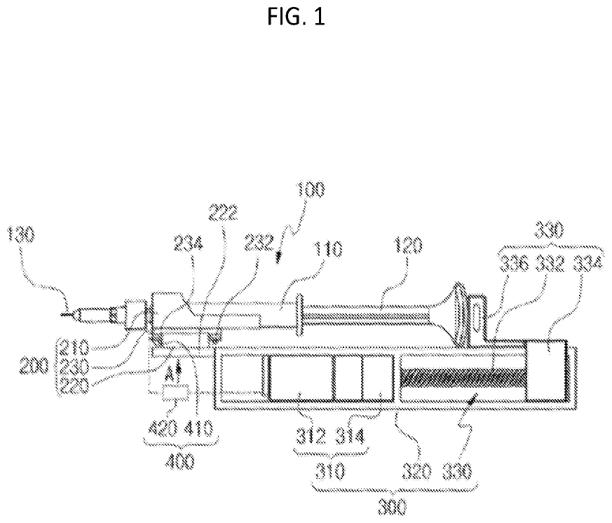

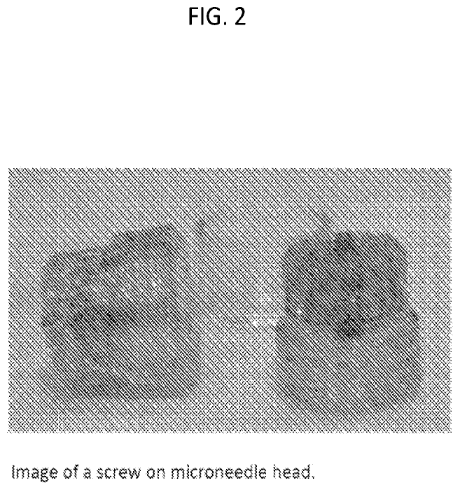

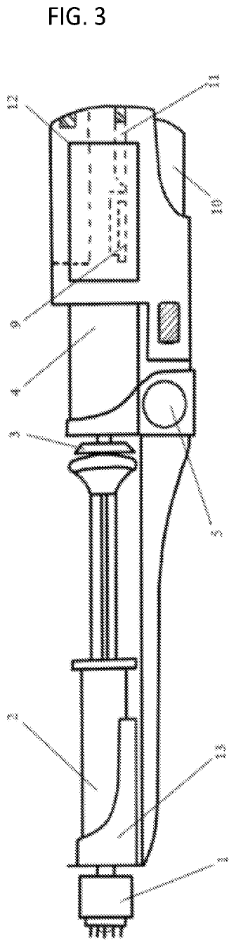

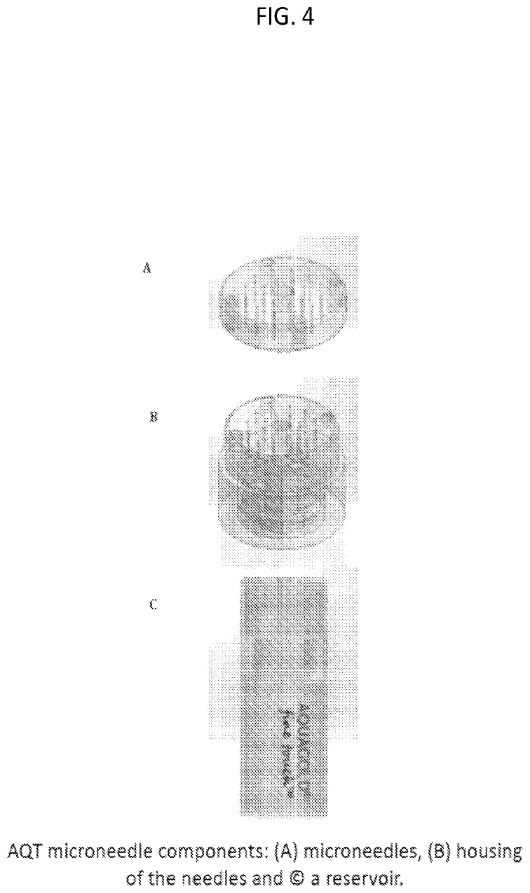

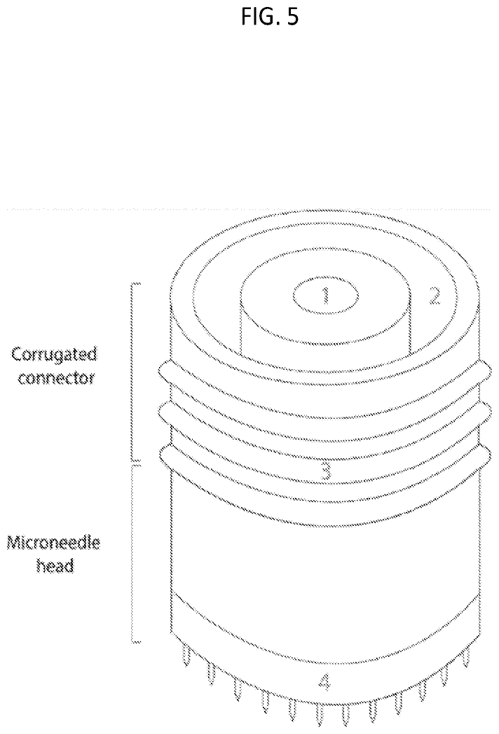

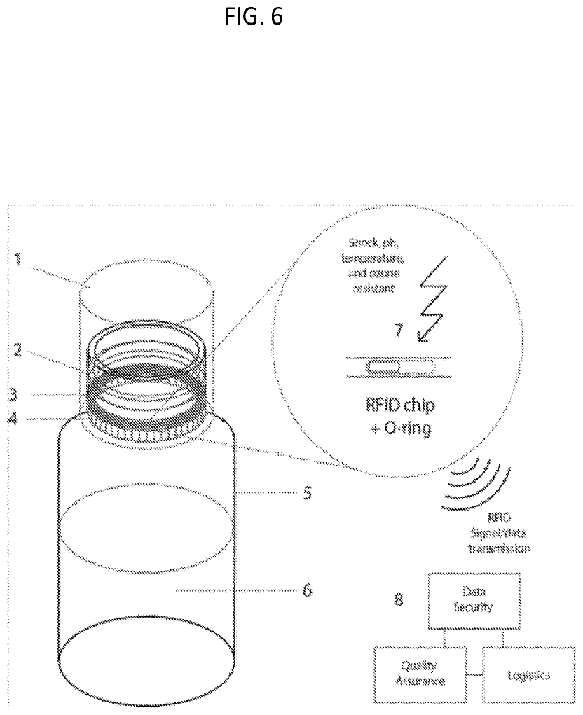

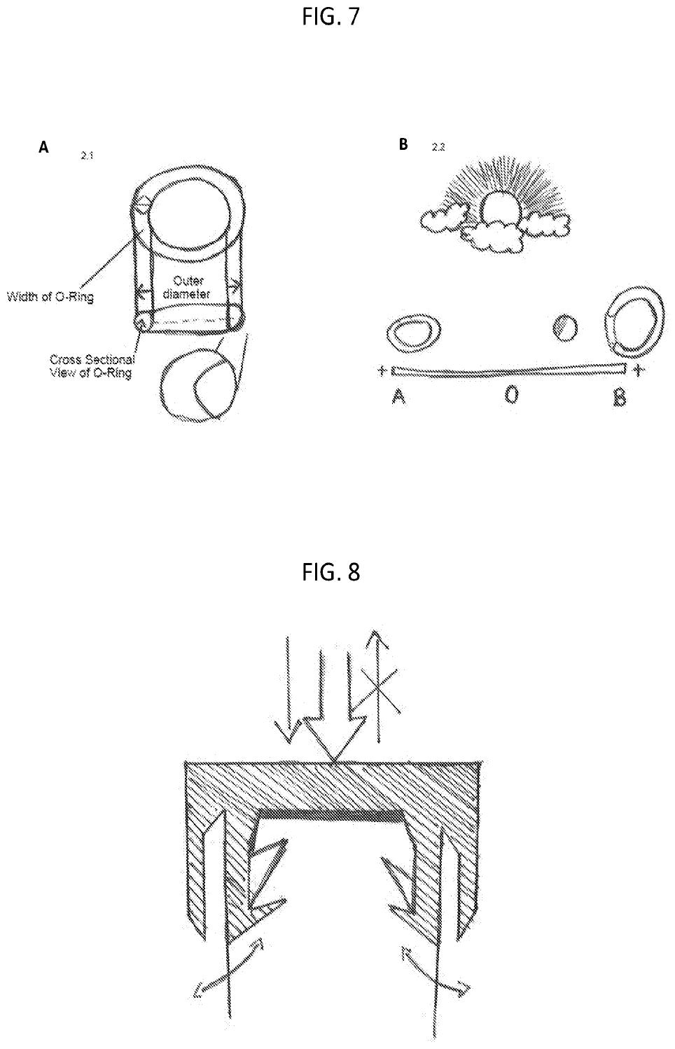

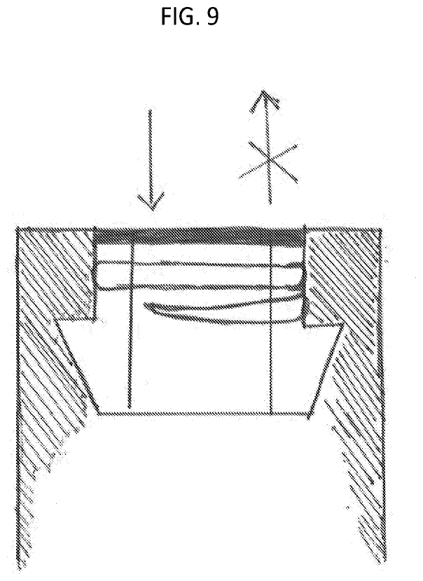

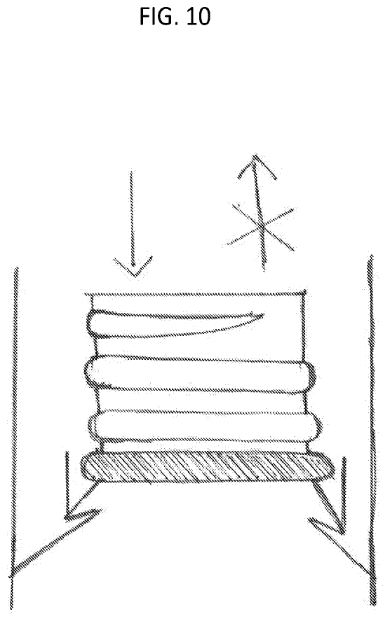

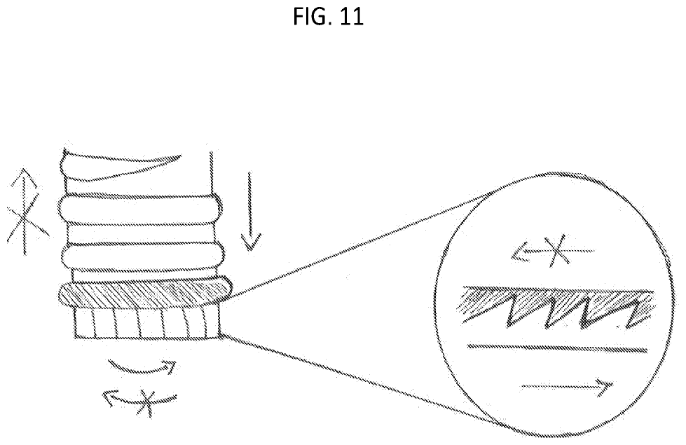

### DETAILED DESCRIPTION

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3rd ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 2%, 3%, 4%, or 5% of a particular term.

### Methods

In one embodiment, the invention provides a method of administering a composition comprising hyaluronic acid to a subject's skin, comprising
i) providing an effective amount of hyaluronic acid in solid form;
ii) reconstituting the hyaluronic acid in an aqueous solution; and
iii) administering the solution comprising hyaluronic acid to the subject's skin.

Generally, the methods include administering an effective amount of a composition as described herein, to a subject who is in need of, or who has been determined to be in need of, such treatment.

A “subject” or “patient” (e.g., a mammal such as a human or a non-human animal) can be a mammal who exhibits one or more clinical manifestations and/or symptoms of a disease or skin condition, hair loss, wound healing and/or prevention of scarring, or for anti-aging, longevity and wellness purpose described herein. In certain situations, a subject may be asymptomatic and yet still have clinical manifestations of the disease or condition.

In one aspect, a formulation is administered until one or more symptoms are improved. In one embodiment, skin elasticity, skin regeneration, metabolism, or a combination thereof are improved by about 2%, about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100% following administration of one or more doses of the formulation to the patient.

In another embodiment, skin elasticity, skin regeneration, metabolism, or a combination thereof are improved by about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 90-fold, about 95-fold, about 100-fold, or more, following administration of one or more doses of the formulation to the patient.

An “effective amount” is an amount sufficient to result in one or more beneficial or desired results, either partially or completely. For example, a therapeutic amount is one that achieves the desired therapeutic effect. For example, a patient may experience about a 10%, 20%, 30%, 40%, 50%, 60%, 70%, 80%, 90%, or 100% improvement in one or more symptoms. This amount can be the same or different from a prophylactically effective amount, which is an amount necessary to improve aging-related skin conditions.

An effective amount can be administered in one or more administrations, applications or dosages. A therapeutically effective amount of a therapeutic compound (i.e., an effective dosage) depends on the aesthetic and therapeutic compounds selected.

One would understand that the compositions provided herein may be administered in the described methods in a variety of dosing regimens which can be determined by the treating physician based upon the patient to be treated and the severity of the condition to be treated. Treatment of a subject with a therapeutically effective amount of the therapeutic compounds described herein can include a single treatment or a series of treatments. For example, a composition may be administered intradermally or topically to a patient on a periodic basis, for example on a daily basis, a weekly basis or as recommended depending on the judgment of the treating physician and the requirements of the individual patient or as recommended. In another example, a composition may be administered once daily for about 5 days, about 10 days, about 20 days, about 30 days, about 2 months, about 3 months, about 4 months, about 5 months, about 6 months, about 1 year, about 2 years, about 3 years, about 4 years, about 5 years or more. Alternatively, a composition may be administered once, twice, three times, about 4 times, about 5 times, about 10 times, about 20 times, about 30 times, about 40 times, about 50 times or more. Alternatively, a composition may be administered on days 1, 3, 7, 10, 14, 21, 30, 60 and 90 of a 90-day treatment period. A patient's symptoms may be monitored during treatment and the physician may alter the treatment schedule based upon one or more of the effects of the compositions.

In one embodiment, a composition described herein is administered 1 day, 2 days, 3 days, 4 days, 5 days or 6 days prior to surgery. In another embodiment, a composition described herein is administered 1 day, 2 days, 3 days, 4 days, 5 days or 6 days after surgery. In yet another embodiment, a composition described herein is administered 1 day, 2 days, 3 days, 4 days, 5 days or 6 days prior to surgery and also 1 day, 2 days, 3 days, 4 days, 5 days or 6 days after surgery

A physician or veterinarian can readily determine and prescribe the effective amount of the formulation required. For example, the physician or veterinarian could start doses of the compounds employed in the formulation at levels lower than that required in order to achieve the desired therapeutic effect and gradually increase the dosage until the desired effect is achieved. Alternatively, a dose can remain constant.

The means by which the solution is administered to the skin is not limiting. In some embodiments, the solution is directly applied to the skin and gently rubbed into the skin. In some embodiments, an applicator device is used to apply the solution to the skin. In some embodiments, the solution is administered to the subject's skin using a microneedle delivery device. In some embodiments, the solution is delivered to the skin with a repeated motion of the microneedle delivery device as provided herein. In some embodiments, the hyaluronic acid in solid form is provided in a reservoir component of a microneedle device. In some embodiments, the hyaluronic acid is reconstituted in the reservoir component.

In some embodiments, the solution is substantially free of preservatives. In some embodiments, the solution has a viscosity that enables efficient injection with a microneedle device.

Hyaluronic acid is provided in solid form. In some embodiments, the HA is in powder form. Hyaluronic acid and sodium hyaluronate can be used interchangeably herein. The hyaluronic acid (HA) is involved in cartilage resilience and skin repair, has been applied medically for decades for a number of different uses including, for example, cartilage resilience and skin repair. Among the most common of these medical applications employ injectable delivery, for example to treat joint pain, or topical delivery, for example to treat dermatitis. Cosmetically, it is often used as an active agent in facial filler injections to smooth wrinkles and in topical creams and gels to rejuvenate the skin and combat the aging process. Hyaluronic acid includes both cross-linked and non-cross-linked hyaluronic acids.

Micro Hyaluronic Acid FCH is highly stable against heat and pH change, exhibits great permeability to stratum corneum and excellent moisturizing property, and can be used as a cosmetic ingredient. Micro Hyaluronic Acid FCH fits comfortably in skin and has excellent permeability to the stratum corneum. Micro Hyaluronic Acid FCH is ultra low viscosity sodium hyaluronate which has an average molecular weight of under 5000. Micro Hyaluronate Acid FCH permeates into skin and shows excellent moisturizing property.

In some embodiments, hyaluronic acid in solid form is crystalline. In some embodiments, it is in powder form. In some embodiments, the hyaluronic acid in solid form is freeze dried. In some embodiments, the hyaluronic acid in solid form is spray-dried. In some embodiments the hyaluronic acid in solid form is vacuum dried. In some embodiments, the hyaluronic acid in solid form is oven dried. In some embodiments, the hyaluronic acid is crosslinked. In some embodiments, the hyaluronic acid is non-crosslinked. In some embodiments, at least a portion of the hyaluronic acid is crosslinked.

In some embodiments, the hyaluronic acid in solid form is in admixture with an effective amount of one or more additional bioactive agents. The bioactive agent is not limiting. In some embodiments, the one or more bioactive agents in admixture can be freeze dried, spray dried, vacuum dried and/or oven dried.

In some embodiments, the hyaluronic acid in solid form or admixture can be in a variety of shapes and is not limiting. In some embodiments, the hyaluronic acid in solid form or admixture is in the shape of flakes, a ball, a cube, or a star, for example.

In some embodiments, the one or more bioactive agents comprises a vitamin, a mineral, retinol, retinoic acid, a bleaching/whitening agent, stem cells, collagen, a neurotoxin, platelet-rich plasma, poly-L-lactic acid, an anesthetic or combinations thereof.

In some embodiments, the concentration of hyaluronic acid following reconstitution comprises an amount of from about 0.25 mg/ml to about 100 mg/ml, 0.5 mg/ml to about 50 mg/ml, 1.0 mg/ml to about 25 mg/ml, from about 2.4 mg/ml to about 12 mg/ml, or from about 4.8 mg/ml to about 7.2 mg/ml.

In some embodiments, the hyaluronic acid is reconstituted in a solution comprising an effective amount of one or more bioactive agents prior to use. In some embodiments, the hyaluronic acid is combined with one or more bioactive agents in solid form and reconstituted. In some embodiments, the reconstituted solution of hyaluronic acid is combined with an effective amount of one or more bioactive agents in solid and/or liquid form. In some embodiments, the one or more bioactive agents in solid form is freeze dried, vacuum dried, spray dried, and/or oven dried. In some embodiments, the one or more bioactive agents in liquid form comprises an aqueous solution.

In some embodiments, the one or more bioactive agents comprises a neuromodulator, vitamin, mineral, PLLA (poly-L-lactic acid) or any combination thereof. In some embodiments, the neuromodulator comprises botulinum toxin (e.g., botulinum toxin of serotype A, B, C, D, E, F or G).

In some embodiments, the methods herein can promote vitality and well-being in a human patient, the method comprising administering a composition of hyaluronic acid as provided herein. This composition or formulation will vary on the process used (cross-linked, non-cross linked). In some embodiments, the solid form of hyaluronic acid that is reconstituted improves viscosity, stability, effectiveness, shelf life and/or other properties, with the goal of ameliorating skin-related symptoms and improving aesthetics.

In some embodiments, the methods herein promote vitality, well-being and improved skin conditions in a human patient. In some embodiments, one or more vitamin B supplement compositions can be co-formulated with the HA herein, e.g., for delivery via a microneedle delivery device.

In some embodiments, there is also provided a method of treating or ameliorating disease or symptoms associated with the aging process in an individual in need thereof, by administering hyaluronic acid as provided herein to the individual. In some embodiments, the composition administered further comprises one or more of cyanocobalamin or/and methylcobalamin; ascorbic acid (vitamin C); ahseutasanchin; Vitamin E; Vitamin D; selenium; zinc; glutathione (GSH); anthocyanidin; Omega-3; EPA; DHA; lecithin; CoQ10; chrome; and magnesium. In one embodiment, the composition further comprises from about 3000 to 5000 mcg cyanocobalamin, from about 3000 to 5000 mcg methylcobalamin, or a combination thereof; from about 700 to about 1000 mg ascorbic acid (vitamin C); from about 11 to about 15 mg zinc; from about 4 to 6 mg ahseutasanchin; from about 100 to 200 IU vitamin E; from about 150 to 300 IU vitamin D; from about 100 to 150 mcg selenium; from about 30 to 50 mg glutathione (GSH); from about 100 to 150 mg anthocyanidin; from about 1 to about 500 mg omega-3; from about 200 to 300 mg EPA; from about 200 to 300 mg DHA; from about 1 to about 500 mg lecithin; from about 30 to 50 mg CoQ10; from about 100 to 150 mcg chrome; from about 150 to 200 mg magnesium.

The disease or symptom to be treated is not limiting and can include those associated with a vitamin B deficiency, such as, for example, chronic fatigue syndrome, high stress levels, pellagra, acne, microcytic anemia, microcytic anemia, reduced skin elasticity, impaired skin regeneration rate, slowed metabolism rate, reduced smoothness and/or softness of skin, dullness of skin, hyperpigmentation or a combination thereof.

Also provided herein is a method of improving tissue repair and regeneration in a human patient in need thereof, comprising administering to the patient an effective amount of a compositions comprising hyaluronic acid as provided herein. In some embodiments, the composition further comprises from about 3000 to 5000 mcg cyanocobalamin, from about 3000 to 5000 mcg methylcobalamin, or a combination thereof; from about 700 to about 1000 mg ascorbic acid (vitamin C); from about 11 to about 15 mg zinc; from about 4 to 6 mg ahseutasanchin; from about 100 to 200 IU vitamin E; from about 150 to 300 IU vitamin D; from about 100 to 150 mcg selenium; from about 30 to 50 mg glutathione (GSH); from about 100 to 150 mg anthocyanidin; from about 1 to about 500 mg omega-3; from about 200 to 300 mg EPA; from about 200 to 300 mg DHA; from about 1 to about 500 mg lecithin; from about 30 to 50 mg CoQ10; from about 100 to 150 mcg chrome; and from about 150 to 200 mg magnesium. In some embodiments, the composition is administered to the patient with a microneedle delivery device.

Also provided herein is a method of improving aging-related skin conditions, such as skin elasticity, skin regeneration, metabolism, smoothness and/or softness of skin (i.e., making the skin feel smoother and softer following treatment); the overall appearance of skin; evening out skin tone and texture; clarity and/or radiance of skin; making the skin look younger; and making wrinkles appear softer and/or less prominent in human patient in need thereof, comprising administering to the patient an effective amount of a compositions comprising hyaluronic acid as provided herein. In some embodiments, the composition further comprises from about 3000 to 5000 mcg cyanocobalamin, from about 3000 to 5000 mcg methylcobalamin, or a combination thereof; from about 700 to about 1000 mg ascorbic acid (vitamin C); from about 11 to about 15 mg zinc; from about 4 to 6 mg ahseutasanchin; from about 100 to 200 IU vitamin E; from about 150 to 300 IU vitamin D; from about 100 to 150 mcg selenium; from about 30 to 50 mg glutathione (GSH); from about 100 to 150 mg anthocyanidin; from about 1 to about 500 mg omega-3; from about 200 to 300 mg EPA; from about 200 to 300 mg DHA; from about 1 to about 500 mg lecithin; from about 30 to 50 mg CoQ10; from about 100 to 150 mcg chrome; and from about 150 to 200 mg magnesium. In some embodiments, the composition is administered to the patient with a microneedle delivery device.

The term “aging-related skin condition” relates to any skin condition or disorder associated with, caused by, or affected by, intrinsic aging and/or extrinsic aging. Aging-related skin conditions that may be treated using the present methods and formulations include, but are not limited to, wrinkles, age spots, sun damage (particularly UV radiation-induced oxidative stress), blemishes, hyperpigmented skin, age spots, increased skin thickness, loss of skin elasticity and collagen content, dry skin, lentigines, melasmas, as well as scars.

Improved skin health, includes but is not limited to improved appearance, increased regeneration, increased elasticity, increased anti-oxidative level, reduced photo-aging, reduced wrinkles, reduced scarring, reduced bacterial activities including acne, redistribution of fat and/or other content of the skin, reduced number and/or sizes of pores, reduced callusing, reduced sweating and/or body odor through skin, improved scalp health, increased hair density, increased evenness of hair growth, and increased hair strength.

### Compositions

In one embodiment, the invention provides a solid composition comprising an effective amount of hyaluronic acid in solid form. In another embodiment, the invention provides a solid composition comprising an effective amount of hyaluronic acid in admixture with an effective amount of one or more bioactive agents.

In some embodiments, the invention provides a reconstituted injectable solution of an effective amount of hyaluronic acid optionally comprising an effective amount of one or more bioactive agents. In some embodiments, the composition has been reconstituted in aqueous solution, wherein the solution has a viscosity that enables efficient injection with a microneedle device.

The compositions can be used in any of the methods and kits of the invention and in combination with any devices, e.g., microneedle devices, provided herein.

In some embodiments, the hyaluronic acid and/or bioactive agent is freeze dried. In some embodiments, the hyaluronic acid and/or bioactive agent is vacuum dried. In some embodiments, the hyaluronic acid and/or bioactive agent is spray-dried. In some embodiments, the hyaluronic acid and/or bioactive agent is oven dried. In some embodiments, the hyaluronic acid is crosslinked. In some embodiments, the hyaluronic acid is non-crosslinked. In some embodiments, at least a portion of the hyaluronic acid is crosslinked.

In some embodiments, the hyaluronic acid in solid form is substantially pure. In some embodiments, the hyaluronic acid is at least about 90%, about 91%, about 92%, about 93%, about 94%, about 95%, about 96%, about 97%, about 98%, about 99%, or about 100% pure. In some embodiments, the hyaluronic acid can contain minimal residual salts, etc., that were derived from the solution that was freeze dried, vacuum dried, etc.

The one or more bioactive agents that can be added is not limiting. In some embodiments, the one or more bioactive agents is elected from the group consisting of one or more vitamins, one or more minerals, retinol, retinoic acid, a bleaching/whitening agent, collagen, a neuromodulator, poly-L-lactic acid, an anesthetic and combinations thereof. In some embodiments, the neuromodulator comprises botulinum toxin (e.g., botulinum toxin of serotype A, B, C, D, E, F or G).

In some embodiments, the one or more bioactive agents that can be added is discussed below.

Vitamins, or vital nutrients, and minerals are not synthesized in the human body and must be obtained from the diet for normal metabolic functioning. While they occur naturally in food, vitamins and minerals are often also taken as oral, injectable, or topical supplements to make up for dietary imbalance or to achieve specific physical effects. The most common vitamins used today to promote skin health are A, B, C, D, and E, while the most common minerals used include zinc and calcium. When referring to a vitamin, it would be understood that all chemical forms of the vitamin are contemplated.

B vitamins are a group of water-soluble vitamins that play important roles in cell metabolism. The B vitamins are B1 (thiamine), B2 (riboflavin), B3 (niacin), B5 (pantothenic acid), B6 (pyridoxine), B7 (biotin), B12 (cobalamin) and folic acid. The B vitamins play an important role in many aspects of the body's functioning, and a vitamin B deficiency can have a serious impact on overall health.

Vitamin B supplements are known in the art: such formulations are limited in terms of absorption (oral dosage forms) or may require a hospital visit (IV therapy) at significant cost in terms of time and expense.

Collagen is a type of fibrous protein found most often in the skin, flesh, and connective tissue of vertebrates. In mammals, it is the most abundant protein in the body, and provides structural support for major tissues and organs. In the skin, it is responsible for providing structure, firmness, and smoothness, and it is often a decrease in collagen production that leads to chronic aging. For this reason, collagen is often injected or topically introduced to the skin in attempts to slow or reverse the effects of aging.

Vitamins and minerals, or vital nutrients, are not synthesized in the human body and must be obtained from the diet for normal metabolic functioning. While they occur naturally in food, vitamins and minerals are often also taken as oral, injectable, or topical supplements to make up for dietary imbalance or to achieve specific physical effects. The most common vitamins used today to promote skin health are A, B, C, D, and E, while the most common minerals used include zinc and calcium.

Vitamin B12, also called cobalamin, is a water-soluble vitamin with a key role in the normal functioning of the brain and nervous system, and for the formation of blood. It is one of the eight B vitamins. It may be involved in the metabolism of every cell of the human body, especially affecting DNA synthesis and regulation, but also fatty acid synthesis and energy production. Vitamin B12 may also be involved in maintenance of the central nervous system and has been used to affect memory loss, Alzheimer's disease, boosting mood, energy and concentration, boost the immune system, and slow aging. Vitamin B12 may also play a role in heart disease, lowering high homocysteine levels (which may contribute to heart disease), male infertility, diabetes, sleep disorders, depression, mental disorders, weak bones (osteoporosis), swollen tendons, AIDS, inflammatory bowel disease, asthma, allergies, a skin disease called vitiligo, preventing cervical and other cancers, and skin infections. Two common forms of Vitamin B12 are cyanocobalamin and methylcobalamin.

Vitamin B12 deficiency may cause macrocytic anemia, fatigue, loss of appetite, loss of balance, weakness, and mood disturbances. It also may cause serious neurologic and neuropsychiatric illness such as paresthesias, ataxia, and memory loss. Vitamin B12 absorption may be impaired at the level of the stomach, where intrinsic factor is produced, or at the level of the terminal ileum, where intrinsic factor bound to vitamin B12 is absorbed.

Niacin and nicotinamide, also known as niacinamide, are forms of vitamin B3. Nicotinamide is the amide of nicotinic acid (vitamin B3/niacin). Nicotinamide is a water-soluble vitamin and is part of the vitamin B group. Nicotinamide may be used for preventing vitamin B3 deficiency and related conditions such as pellagra. Each of these forms of vitamin B3 may be used for schizophrenia, hallucinations due to drugs, Alzheimer's disease and age-related loss of thinking skills, chronic brain syndrome, depression, motion sickness, alcohol dependence, and fluid collection (edema).

Vitamin B1, also known as thiamine, is a water-soluble vitamin and may be utilized for metabolizing carbohydrates and production of energy. Vitamin B1 also may aid in the function of the heart and cardiovascular system and the nervous system.

Vitamin B6, also known as pyridoxine, may be involved in many aspects of macronutrient metabolism, neurotransmitter synthesis, histamine synthesis, hemoglobin synthesis and function and gene expression. Vitamin B6 may assist with cellular metabolism, supports the immune system, with formation of red blood cells and maintenance of healthy brain function. Vitamin B6 may be used for Alzheimer's disease, attention deficit-hyperactivity disorder (ADHD), Down syndrome, autism, diabetes and related nerve pain, sickle cell anemia, migraine headaches, asthma, carpal tunnel syndrome, night leg cramps, muscle cramps, arthritis, allergies, acne and various other skin conditions, and infertility. It is also may be used to treat dizziness, motion sickness, preventing the eye disease age-related macular degeneration (AMD), seizures, convulsions due to fever, and movement disorders (tardive dyskinesia, hyperkinesis, chorea), as well as for increasing appetite and helping people remember dreams. Vitamin B6 may be used for acne, leprosy, attention deficit-hyperactivity disorder (ADHD), memory loss, arthritis, preventing premenstrual headache, improving digestion, protecting against toxins and pollutants, reducing the effects of aging, lowering blood pressure, improving circulation, promoting relaxation, improving orgasm, and preventing cataracts. Vitamin B6 deficiency may cause anemia due to insufficient production of hemoglobin.

Vitamin B2, also known as riboflavin, releases energy from carbohydrates and may be used for preventing low levels of riboflavin (riboflavin deficiency), cervical cancer, and migraine headaches. It also may be used for treating riboflavin deficiency, acne, muscle cramps, burning feet syndrome, carpal tunnel syndrome, and blood disorders such as congenital methemoglobinemia and red blood cell aplasia. It also may be used for increasing energy levels; boosting immune system function; maintaining healthy hair, skin, mucous membranes, and nails; slowing aging; boosting athletic performance; promoting healthy reproductive function; canker sores; memory loss, including Alzheimer's disease; ulcers; burns; alcoholism; liver disease; sickle cell anemia; and treating lactic acidosis brought on by treatment with a class of AIDS medications called NRTI drugs.

The term “vitamin B6” encompasses multiple forms of vitamin B6 suitable for human administration. Several forms of the vitamin are known, but pyridoxal phosphate (PLP; “pyridoxine”) is the active form and may be used as a cofactor in many reactions of amino acid metabolism, including transamination, deamination, and decarboxylation. Pyridoxine may be used in enzymatic reactions affecting the release of glucose from glycogen.

Vitamin C, also known as ascorbic acid, is an antioxidant. Vitamin C may be used to protect against free radicals and promote a healthy immune system, wound healing, and forming healthy skin. More specifically, ascorbic acid may be used to prevent and treat scurvy, a disease caused by a lack of vitamin C in the body. People with high intakes of vitamin C from fruits and vegetables may have a lower risk of getting many types of cancer, such as lung, breast, and colon cancer.

Vitamin B5, also known as pantothenic acid, has skincare benefits. For example, it increases the degree of hydration of the skin, reduces the trans-epidermal water loss and keeps the elasticity and smoothness of the skin. Vitamin B5 may be used in acne treatments and may be used to reduce itchiness of the skin.

Zinc is an essential mineral found in cells throughout the body. Zinc is required for protein synthesis and collagen formation, and may be used to promote a healthy immune system and assist in wound healing. It may also be used for muscular growth and contraction and to protect the liver from chemical damage such as which can occur with anesthetics or other drugs or toxins. Zinc may also be utilized in bone formation. Zinc deficiency may contribute to fatigue, susceptibility to infection, and slow wound healing.

Ahseutasanchin is a unique pigment belonging to the carotenoid family. It exhibits antioxidant capacity against free radicals.

Vitamin E can boost the immune system and protect people against toxins such as air pollution, neurological disease such as Alzheimer's disease, and diabetes. As an antioxidant, Vitamin E can remove free radicals that damage the cell structure. Owing to this property, another well-known health benefit for Vitamin E is in skin and hair care.

Vitamin D helps intestines absorb nutrients and is essential for calcium utilization, ensuring strong bones and robust immune system.

Selenium displays antioxidant properties that regenerate vitamin C and vitamin E, thereby decreasing the aging of the skin and protecting cells from damage. Moreover, selenium also benefits the immune system and protects our body against various infections.

Glutathione (GSH) is an antioxidant which prevents damage to important cellular components caused by free radicals. The strong antioxidant effect of glutathione helps keep cells running smoothly and it also helps the liver remove chemicals that are foreign to the body, such as drugs and pollutants.

Anthocyanidins have a wide range of biological activities including antioxidant, anti-inflammatory, antimicrobial and anti-cancer activities. In addition they display a variety of effects on blood vessels, platelets and lipoproteins able to reduce the risk of coronary heart diseases.

EPA is a form of omega-3 fatty acids which can reduce cellular inflammation.

DHA is a building block of tissue in the brain and retina of the eye. It helps with forming neural transmitters, such as phosphatidylserine, which is important for brain function. EPA and DHA are also well-known for improving skin conditions. Their anti-inflammatory properties help prevent various skin ailments. EPA and DHA can also reduce the damage caused by overexposure to the sun and negative impacts of UV rays.

Lecithin acts as a solvent for cholesterol, triglycerides, and other fats. Therefore, it helps to prevent such ailments as high blood pressure, stroke, heart disease, hardening of the arteries, etc. Also, lecithin plays a vital role in the absorption of nutrients out of the blood stream into the cells.

CoQ10 helps to combat fatigue, boosts immune system, fight against free radicals, and keep cells both inside the body and in the skin healthy. The CoQ10 level decreases as people get older, resulting in an impeded ability to produce collagen and elastin, and the loss of collagen and elastin causes our skin wrinkle and sag.

Magnesium can benefit blood pressure and help prevent sudden cardiac arrest and stroke. It also plays a role in detoxification processes and therefore is important for helping to prevent damage from environmental chemicals, heavy metals and other toxins.

Vitamins and minerals, or vital nutrients, are not synthesized in the human body and must be obtained from the diet for normal metabolic functioning. While they occur naturally in food, vitamins and minerals are often also taken as oral, injectable, or topical supplements to make up for dietary imbalance or to achieve specific physical effects. The most common vitamins used today to promote skin health are A, B, C, D, and E, while the most common minerals used include zinc and calcium.

Bleaching/whitening agents that may be used in the compositions described herein include, but are not limited to, hydroquinone, kojic acid, ascorbic acid, magnesium ascorbyl phosphate or ascorbyl glucosamine, hydroquinone, licorice extract (e.g., Glycyrrhiza glabra (licorice) root extract), an alpha MSH antagonist (e.g. undecylenoyl phenylalanine), phytic acid, monobenzyl ether of hydroquinone, azelaic acid, kojic acid, mequinol, retinoids (e.g., tretinoin, adapalene), soy proteins, alpha-hydroxy acids (e.g., glycolic acid), trichloroacetic acid, salicylic acid, hydroquinone-beta-D-glucopyranoside, paper mulberry, glabridin, 4-isopropylcetchol, aleosin, N-acetyl-4-S-cycteaminylphenol, N-propionyl-4-S-cysteaminylphenol, N-acetyl glucosamine, tranexamic acid and mixtures thereof.

Retinols used in the compositions described herein include, but are not limited to, retinoic acid.

Collagen is a type of fibrous protein found most often in the skin, flesh, and connective tissue of vertebrates. In mammals, it is the most abundant protein in the body, and provides structural support for major tissues and organs. In the skin, it is responsible for providing structure, firmness, and smoothness, and it is often a decrease in collagen production that leads to chronic aging. For this reason, collagen is often injected or topically introduced to the skin in attempts to slow or reverse the effects of aging (Varani J, Dame M K, Rittie L, Fligiel S E G, Kang S, Fisher G J, Voorhees J J: Decreased Collagen Production in Chronically Aged Skin. Am J Pathol. 2006 June; 168(6):1861-1868. PubMed PMCID: PMC1606623).

Botulinum toxin, a neurotoxic protein, is used cosmetically and therapeutically for treatment of facial lines and wrinkles, upper motor neuron syndrome, excessive sweating, cervical dystonia, chronic migraine, and overactive bladder. The toxin is generally injected into the subcutaneous muscles at the target areas, and works by temporarily (for a period of six weeks to eight months, depending on the location and the dose) inhibiting the release of acetylcholine at the neuromuscular junction and thus paralyzing the muscles achieve the desired affects (BOTOX (onabotulinumtoxinA) [prescribing information]. Irvine, Calif. Allergan, Inc. January 2013). Botulinum toxin refers to any botulinum toxin, including but not limited to botulinum toxin type A, botulinum toxin type B, botulinum toxin type Cl, botulinum toxin type D, botulinum toxin type E, botulinum toxin type F and botulinum toxin type G. Botulinum toxin type A includes, for example, Botox, Dysport and Xeomin. Botulinum toxin type B includes, for example, MyoBloc. Botulinum toxin may be provided in a liquid or powder form. A powdered form may be, for example, a sterile, lyophilized preparation. Lyophilized preparations may be reconstituted prior to application. Alternatively, botulinum toxin may be provided as a sterilized pre-dissolved solution. Botulinum toxin may be formulated in an amount of about 0.01 to about 60 units.

“MicroBotox” or “Purtox” as used herein, refers to instances when diluted Botox is injected in multiple very small doses in a treated area. The effects of the Botox are more evenly spread over the areas treated and the risks of having areas over-treated is reduced. Use of MicroBotox generally results in a more natural look (i.e., less frozen) and the dosage of Botox administered is reduced. For some patients suffering from recalcitrant acne problems, MicroBotox (referred to as “mesoBotox” when used in this situation) can be injected very superficially into the facial skin. Following dilution, microBotox may be formulated in an amount of 0.1 to about 99% of the compositions. For example, one would use 0.1 to about 100 units of onabotulinum toxin diluted with at least 2.5 cc of saline.

Minoxidil is a vasodilator that was originally administered orally as a treatment for hypertension, but was found to have the additional effect of slowing hair loss and promoting hair growth. It is now a common topical treatment for androgenic hair loss, and is thought to achieve hair regrowth by increasing the blood flow (and thus the availability of oxygen and vital nutrients) to the hair follicles, stimulating them to resume normal functioning (Olsen E A, Whiting D, Bergfeld W, Miller J, Hordinsky M, Wanser R, Zhang P, Kohut B: A multicenter, randomized, placebo-controlled, double-blind clinical trial of a novel formulation of 5% minoxidil topical foam versus placebo in the treatment of androgenetic alopecia in men. J Am Acad Dermatol. 2007 Aug. 29. PubMed PMID: 17761356).

Platelet-rich plasma (PRP) is blood plasma that has been enriched by platelets, and is prepared by separating whole blood via centrifugation and then collecting the plasma-rich layers that emerge. Because it has five times the baseline platelet concentration of plasma (.about.100,000 platelets per microliter as opposed to the baseline of .about.20,000 platelets per microliter), it contains a number of different growth factors (proteins that stimulate tissue growth, the release of which can be induced by the addition of thrombin and calcium chloride. PRP injections have been used clinically for several years as a treatment for nerve, bone, and muscle injuries, and have been used cosmetically to reverse damage to the skin and to promote dermal strength and rejuvenation (Borrione P, Gianfrancesco A D, Pereira M T, Pigozzi F: Platelet-rich plasma in muscle healing. Am J Phys Med Rehabil. 2010 October; 89(10):854-61. PubMed PMID: 20855985).

Poly-L-lactic Acid (PLLA) is a type of dermal filler used in the treatment of facial lipoatrophy (the gradual loss of facial fat, generally due to aging). PLLA, upon entering the skin, provides immediate structural support to the skin and also promotes the neosynthesis of collagen, hiding sunken areas. Over time, it is converted by the body into harmless lactic acid, gradually transferring the load to the recently synthesized collagen (SCULPTRA Aesthetic (injectable poly-L-lactic acid) [prescribing information]. Bridgewater, N.J. Sanofi-Aventis U.S. LLC. May 2012).

Bimatoprost is a prostaglandin prodrug that is administered topically to control the progression of Glaucoma and to treat ocular hypertension. Since 2008, the application of this drug has evolved to encompass a cosmetic formulation for the lengthening and darkening of eyelashes and is thought to confer an improved appearance by delivering bimatoprost—a growth stimulating analog—circambient to the hair follicles at the edge of the eyelid.

Compositions described herein may be used to treat one or more clinical manifestations and/or symptoms of a disease or skin condition, hair loss, wound healing and/or prevention of scarring, or for anti-aging, longevity and wellness purposes described herein. The present inventors have identified new compositions that may be injected into a patient to improve skin elasticity, skin regeneration, metabolism, smoothness and/or softness of skin (i.e., making the skin feel smoother and softer following treatment); the overall appearance of skin; evening out skin tone and texture; clarity and/or radiance of skin; making the skin look younger; and making wrinkles appear softer and/or less prominent, etc.

In some embodiments, the compositions and methods provided herein provide compositions formulated for administration to the skin surface in combination with a microneedle device, the compositions being applied topically, intradermally or a combination of both routes of administration. The compositions and methods provided herein provide vitamin compositions formulated for administration to the skin surface in combination with a microneedle device, the compositions being applied percutaneously. Suitable amounts can be determined by the skilled physician and depend on the age, gender and general health of the patient, as well as a surgical, or other procedure planned for the patient. In some cases, it may also be determined based upon the degree and type of trauma a patient has suffered. Previous nutritional status may also be considered when determining a suitable amount of nutritional supplementation. In addition, patient compliance is a very important factor, since nutritional supplementation is not effective if the patient does not receive the supplements and is much less effective if the patient does not receive the proper dose on a consistent basis.

Improved skin health or improved skin quality, as used above, includes but is not limited to improved appearance, increased regeneration, increased elasticity, increased anti-oxidative level, reduced photo-aging, reduced wrinkles, reduced scarring, reduced bacterial activities including acne, redistribution of fat and/or other content of the skin, reduced number and/or sizes of pores, reduced callusing, reduced sweating and/or body odor through skin, improved scalp health, increased hair density, increased evenness of hair growth, and increased hair strength.

The compositions can be provided in easy-to-use and clearly labeled packs to increase patient compliance. The compositions may be administered to any patient needing, e.g., improvement of a skin condition such as, for example, skin elasticity, skin regeneration, metabolism, etc. For example, patient populations include, but are not limited to: men over age fifty, and women over age fifty. That is, patients whose skin may not be as elastic or firm as a younger patient. However, it would be understood that humans under the age of fifty may also experience changes in their skin which would benefit from administration of a composition described herein. For example, a patient who may also be administered a composition described herein may be one who had plastic or reconstructive surgery, which population includes any human of any age. In addition to these compositions, a treating physician may add other disease-specific supplements as the patient's condition warrants. In addition to the pre-packaged nutritional supplements, the dispensing physician may add one or more other specific supplements, if needed.

Compositions comprising hyaluronic acid in combination with botulinum toxin, collagen, vitamins, minerals, biamptoprost and/or minoxidil act on the skin and/or subcutaneous muscles in order to boost cell rejuvenation and creation, alleviate fine lines and wrinkles, reduce the appearance of scars and blemishes, and improve skin clarity, elasticity, firmness, tone, vitality, and overall health.

In some embodiments, the one or more bioactive agents include, but are not limited to, B1 (thiamine), B2 (riboflavin), B3 (niacin), B5 (pantothenic acid), B6 (pyridoxine), B7 (biotin), B12 (cobalamin), collagen, botulinum toxin, platelet-rich plasma, poly-L-lactic acid, lidocaine, epinephrine and/or folic acid.

In one embodiment, the solid composition comprises a therapeutically effective amount of one or more of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, (HA), collagen, botulinum toxin (e.g., botulinum toxin of serotype A, B, C, D, E, F or G), platelet-rich plasma (PRP), poly-L-lactic acid (PLLA), and optionally lidocaine with epinephrine, a chemical stabilizer and optionally a preservative, wherein the composition can be reconstituted into an aqueous solution and is formulated for topical use and/or dermal administration. The pH of the solution can be adjusted to a pH that is physiologically acceptable for administration to humans. In one embodiment, a composition comprising HA contains only three of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, collagen, botulinum toxin (e.g., botulinum toxin of serotype A, B, C, D, E, F or G), platelet-rich plasma, poly-L-lactic acid and/or lidocaine with epinephrine. In another embodiment, the HA composition contains only four of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, collagen, botulinum toxin (e.g., botulinum toxin of serotype A, B, C, D, E, F or G), platelet-rich plasma, poly-L-lactic acid and/or lidocaine with epinephrine. In another embodiment, the HA composition contains only five of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, collagen, botulinum toxin (e.g., botulinum toxin of serotype A, B, C, D, E, F or G), platelet-rich plasma, poly-L-lactic acid and/or lidocaine with epinephrine. In another embodiment, the HA composition contains only six of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, collagen, botulinum toxin (e.g., botulinum toxin of serotype A, B, C, D, E, F or G), platelet-rich plasma, poly-L-lactic acid and/or lidocaine with epinephrine. In another embodiment, the HA composition contains all of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, hyaluronic acid, collagen, botulinum toxin (e.g., botulinum toxin of serotype A, B, C, D, E, F or G), platelet-rich plasma, poly-L-lactic acid and lidocaine with epinephrine.

Vitamin B12 may be included in a composition in amount to improve skin appearance. In one embodiment, the aqueous solution comprises from about 2500 to about 3000 mcg cobalamin, from about 3500 to about 4500 mcg cobalamin, or from about 4000 to about 5000 mcg cobalamin. In one embodiment, vitamin B12 is present in a composition in an amount of about 5000 mg. One would appreciate that vitamin B12 exists in multiple forms suitable for therapeutic use, including cyanocobalamin and methylcobalamin, which forms are contemplated for use herein.

Vitamin B3 (nicotinamide) may be included in a composition in amount of from about 10 to about 60 mg, from about 20 to about 50 mg, from or about 30 to about 40 mg. In one embodiment, thiamine is present in a composition in an amount of about 40 mg.

Vitamin B1 may be included in a composition in an amount of from about 2 to about 10 mg, from about 4 to 8 mg, or from about 4 to about 6 mg. In one embodiment, thiamine is present in a composition in an amount of about 6 mg.

Vitamin B6 (pyridoxine) may be included in a composition in an amount of from about 0 to about 0.2 mg, about 0 to 0.1 mg, or about 0.1 to about 0.2 mg. Pyridoxine may be administered as pyridoxine HCl. In one embodiment, pyridoxine HCl is present in a composition in an amount of about 0.1 mg.

Vitamin B2 (Riboflavin 5-phosphate sodium) may be included in a composition in an amount of from about 2 to about 10 mg, from about 4 to about 8 mg or from about 4 to about 6 mg. In one embodiment, riboflavin 5-phosphate sodium is present in a composition in an amount of about 3.6 mg.

Vitamin C (ascorbic acid) may be included in a composition in an amount of from about 50 to about 300 mg, from about 100 to about 250 mg, or from about 150 to about 200 mg. In one embodiment, ascorbic acid is present in a composition in an amount of about 200 mg.

Zinc sulfate heptahydrate may be included in a composition in an amount of from about 0 to about 0.2 mg, or from about 0 to about 0.1 mg. In one embodiment, zinc is present in a composition in an amount of about 0.1 mg.

Collagen, botulinum toxin (e.g., botulinum toxin of serotype A, B, C, D, E, F or G), platelet-rich plasma, and poly-L-lactic acid may be included in a composition in any amount as considered necessary to achieve the aesthetic goals set by the patient and doctor.

In some embodiments, the reconstituted aqueous solution may optionally include lidocaine with epinephrine, which are used for providing patient comfort and reducing bruising.

In some embodiments, the reconstituted aqueous solution may optionally include a chemical stabilizer. Any number of chemical stabilizers may be used to stabilize and increase shelf-life of formulations. In one aspect, a chemical stabilizer is employed to retard or prevent degradation of a vitamin compound induced by ultraviolet light. Chemical stabilizers include, but are not limited to, a chelating agent, an antioxidant, an acidifying agent or gentisic acid. In one embodiment, the chemical stabilizer is an acidifying agent or gentisic acid. In some embodiments, the reconstituted aqueous does not comprise a chemical stabilizer or other preservative.

Solution formulations can be prepared by dissolving these components in an aqueous solvent such as a buffered aqueous solution known in the art of solution formulations such as phosphate buffers and/or citrate buffers. Unbuffered saline or water can also be used.

Provided herein is a reconstituted HA aqueous solution adapted for human administration, comprises an effective amount of HA, from about 3000 to 5000 mcg cyanocobalamin, from about 3000 to 5000 mcg methylcobalamin, or a combination thereof; from about 700 to about 1000 mg ascorbic acid (vitamin C); from about 11 to about 15 mg zinc; from about 4 to about 6 mg ahseutasanchin; from about 100 to about 200 IU vitamin E; from about 150 to about 300 IU vitamin D; from about 100 to about 150 mcg selenium; from about 30 to about 50 mg glutathione (GSH); from about 100 to about 150 mg anthocyanidin; from about 1 to about 500 mg omega-3; from about 200 to about 300 mg EPA; from about 200 to about 300 mg DHA; from about 1 to about 500 mg lecithin; from about 30 to about 50 mg CoQ10; from about 100 to about 150 mcg chrome; from about 150 to about 200 mg magnesium; optionally a chemical stabilizer; and optionally a preservative, wherein the aqueous solution is formulated for administration via a microneedle device.

In one embodiment, the reconstituted HA aqueous solution comprises an effective amount of HA, about 5000 mcg cyanocobalamin and/or methylcobalamin; about 1000 mg ascorbic acid (vitamin C); about 15 mg zinc; about 6 mg ahseutasanchin; about 200 IU vitamin E; about 300 IU vitamin D; about 150 mcg selenium; about 50 mg glutathione (GSH); about 150 mg anthocyanidin; about 500 mg omega-3; about 300 mg EPA; about 300 mg DHA; about 500 mg lecithin; about 50 mg CoQ10; about 150 mcg chrome; and about 200 mg magnesium.

In one embodiment, a chemical stabilizer for use in the aqueous solution is gentisic acid. A chemical stabilizer may be present in the formulation in an amount of from about 0.01% to about 2%.

In one embodiment, a preservative is benzyl alcohol. A preservative may be present in the formulation in an amount of from about 0.01% to about 2%. In some embodiments, no preservative is needed.

In one embodiment, the product has a pH from about 7.2 and 7.6, for example, a pH of 7.4.

In one embodiment, the cobalamin is selected from cyanocobalamin or methylcobalamin.

Also provided herein is a reconstituted HA aqueous solution formulated for administration via microneedle device comprising an effective amount of HA, from about 3000 to 5000 mcg cyanocobalamin, from about 3000 to 5000 mcg methylcobalamin, or a combination thereof; from about 700 to about 1000 mg ascorbic acid (vitamin C); from about 11 to about 15 mg zinc; from about 4 to about 6 mg ahseutasanchin; from about 100 to about 200 IU vitamin E; from about 150 to about 300 IU vitamin D; from about 100 to about 150 mcg selenium; from about 30 to about 50 mg glutathione (GSH); from about 100 to about 150 mg anthocyanidin; from about 1 to about 500 mg omega-3; from about 200 to about 300 mg EPA; from about 200 to about 300 mg DHA; from about 1 to about 500 mg lecithin; from about 30 to about 50 mg CoQ10; from about 100 to about 150 mcg chrome; and from about 150 to about 200 mg magnesium. The aqueous solution may further comprise a chemical stabilizer. The aqueous solution may further comprise a preservative. The aqueous solution may have a pH of about 7.2 to about 7.6 such as, for example, a pH of 7.4.

The compositions may be sterile and should be fluid to the extent that easy injectability exists. It should be stable under the conditions of manufacture and storage and can be preserved against the contaminating action of microorganisms such as bacteria and fungi.

Reconstituted HA aqueous solutions can be prepared by incorporating the aforementioned components in the required amount in an appropriate solvent with one or a combination of ingredients enumerated above, as required. In some embodiments, the solutions can be filter sterilized, UV sterilized, subjected to gamma irradiation, e-beam sterilization, or any other conventional method for sterilization of fluids. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof. The pH of the solution can be adjusted to a pH that is physiologically acceptable for administration to humans.

In one embodiment, the reconstituted HA aqueous solutions adapted for human administration comprises an effective amount of HA, from about 1500 to about 5000 mcg cobalamin (vitamin B12); from about 100 to about 200 mg ascorbic acid (vitamin C); from about 10 to about 60 mg nicotinamide (vitamin B3); from about 2 to about 10 mg thiamine hydrochloride (vitamin B1); from about 0.1 to about 0.25 mg pyridoxine HCl (vitamin B6); from about 2 to about 10 mg riboflavin 5-phosphate sodium (vitamin B2); from about 0.1 to about 0.5 mg zinc sulfate heptahydrate; from about 2.4 to about 12 mg/ml HA; optionally lidocaine with epinephrine; optionally a chemical stabilizer; and optionally a preservative. Preservatives and chemical stabilizers may be added to the compositions in an amount of from about 0.01% and about 2% of the total volume.

In one embodiment, the reconstituted HA aqueous solutions adapted for human administration comprises an effective amount of HA (e.g., about 12 mg/ml HA), about 5000 mcg cobalamin (vitamin B12); about 200 mg ascorbic acid (vitamin C); about 40 mg nicotinamide (vitamin B3); about 6 mg thiamine (vitamin B1); about 0.1 mg pyridoxine HCl (vitamin B6); about 3.6 mg riboflavin 5-phosphate sodium (vitamin B2); about 0.1 mg zinc sulfate heptahydrate, and any concentration of, collagen, botulinum toxin (e.g., botulinum toxin of serotype A, B, C, D, E, F or G), platelet-rich plasma (PRP), and/or poly-L-lactic acid (PLLA) as determined necessary to achieve aesthetic goals set by the doctor and patient. A product provided herein may, in some instances, in addition to HA contain three, four, five, six, or all of the following: cobalamin (vitamin B12); ascorbic acid (vitamin C); nicotinamide (vitamin B3); thiamine (vitamin B1); pyridoxine HCl (vitamin B6); riboflavin 5-phosphate sodium (vitamin B2); zinc sulfate heptahydrate; collagen, botulinum toxin (e.g., botulinum toxin of serotype A, B, C, D, E, F or G), platelet-rich plasma (PRP), and/or poly-L-lactic acid (PLLA).

In one embodiment, the reconstituted HA aqueous solutions adapted for human administration comprises an effective amount of HA (e.g., about 12 mg/ml HA) and one or more of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, collagen, botulinum toxin, platelet-rich plasma (PRP), poly-L-lactic acid (PLLA), and optionally lidocaine with epinephrine, a chemical stabilizer and optionally a preservative, wherein the aqueous solution is formulated for delivery with a microneedle device. The pH of the solution can be adjusted to a pH that is physiologically acceptable for administration to humans.

In another embodiment, the reconstituted HA aqueous solutions adapted for human administration comprises an effective amount of HA (e.g., about 12 mg/ml HA), and only three of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, collagen, botulinum toxin, platelet-rich plasma, poly-L-lactic acid and/or lidocaine with epinephrine.

In another embodiment, the reconstituted HA aqueous solutions adapted for human administration comprises an effective amount of HA (e.g., about 12 mg/ml HA), and only four of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, collagen, botulinum toxin, platelet-rich plasma, poly-L-lactic acid and/or lidocaine with epinephrine.

In one embodiment, the reconstituted HA aqueous solutions adapted for human administration comprises an effective amount of HA (e.g., about 12 mg/ml HA), and only five of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, collagen, botulinum toxin, platelet-rich plasma, poly-L-lactic acid and/or lidocaine with epinephrine.

In one embodiment, the reconstituted HA aqueous solutions adapted for human administration comprises an effective amount of HA (e.g., about 12 mg/ml HA), and contains only six of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, collagen, botulinum toxin, platelet-rich plasma, poly-L-lactic acid and/or lidocaine with epinephrine.

In another embodiment, a composition contains all of the following: cobalamin (vitamin B12), ascorbic acid (vitamin C), nicotinamide (vitamin B3), thiamine (vitamin B1), pyridoxine HCl (vitamin B6), riboflavin 5-phosphate sodium (vitamin B2), zinc sulfate heptahydrate, hyaluronic acid, collagen, botulinum toxin (e.g., botulinum toxin of serotype A, B, C, D, E, F or G), platelet-rich plasma, poly-L-lactic acid and lidocaine with epinephrine.

In another embodiment, the reconstituted HA aqueous solutions adapted for human administration comprises an effective amount of HA (e.g., about 12 mg/ml HA), and from about 3000 to 5000 mcg cyanocobalamin, from about 3000 to 5000 mcg methylcobalamin, or a combination thereof; from about 700 to about 1000 mg ascorbic acid (vitamin C); from about 11 to about 15 mg zinc; from about 4 to 6 mg ahseutasanchin; from about 100 to 200 IU vitamin E; from about 150 to 300 IU vitamin D; from about 100 to 150 mcg selenium; from about 30 to 50 mg glutathione (GSH); from about 100 to 150 mg anthocyanidin; from about 1 to about 500 omega-3; from about 200 to 300 mg EPA; from about 200 to 300 mg DHA; from about 1 to about 500 mg lecithin; from about 30 to 50 mg coenzyme Q10 (CoQ10); from about 100 to 150 mcg chrome; from about 150 to 200 mg magnesium; optionally a chemical stabilizer; and optionally a preservative, wherein the aqueous solution is formulated for delivery using microneedle devices. In order to achieve personalized benefits at a maximal level, one may add or remove few of the components listed above, as well as increasing or reducing the dose. Preservatives and chemical stabilizers may be added to the compositions in an amount of from about 0.01% and about 2% of the total volume.

In another embodiment, the reconstituted HA aqueous solutions adapted for human administration comprises an effective amount of HA (e.g., about 12 mg/ml HA) and micro botulinum toxins alone or with other component combinations whereby certain botulinum toxin can be freeze- or vacuum-dried and pre-filled in an container and a user reconstitutes and dilutes an injectable dose of the said botulinum toxin into solution, e.g., saline; and/or whereby a liquid product of botulinum toxin is diluted with reconstituted HA aqueous solutions. In some embodiments, from 0.2 to 3 ml per up to 49 units of botulinum toxin alone or combined with hyaluronic acid.

In some embodiments, the reconstituted HA aqueous solutions are formulated in physiological saline and adjusted to an acceptable pH in the range of about 6.5 to about 7.5, thereby neutralizing the solution to minimize pain upon transdermal delivery. Such an aqueous formulation may be optionally supplemented with a preservative (e.g., from about 0.01% to about 2% benzyl alcohol) and/or a chemical stabilizer (e.g., from about 0.01% to about 2% gentisic acid), depending on the anticipated shelf life and environmental factors that could lead to degradation. Painless delivery allows for exclusion of lidocaine or other similar compounds to reduce pain or have an anesthetic effect before non-invasive laser treatments.

In some embodiments the reconstituted HA aqueous solution comprises hyaluronic acid (cross-linked and/or non-cross-linked or combinations of both) alone or in combination with a combination of micro botulinum toxin and/or vitamins.

A combination therapeutics may be applied to traditional areas of treatment for aesthetic application including the forehead, neck, lower eyelids, cheek, periorbitally, and intraorbitally as well as non-traditional areas such as decollate, dorsum of hand, knees, elbows, ankles, lower buttocks, lower part of breasts, chin etc. Age-related skin features such as brow and frown lines, crow's feet, and bunny lines may be targeted to achieve aesthetic goals.

In some embodiments the reconstituted HA aqueous solution has a volume suitable for microinjection to a subject's skin. In some embodiments volumes formulated for microinjection include, but are not limited to, about 0.1 ml, about 0.2 ml, about 0.3 ml, about 0.4 ml, about 0.5 ml, about 0.6 ml, about 0.7 ml, about 0.8 ml, about 0.9 ml, about 1.0 ml or above.

The solid compositions can be provided in a container and/or reconstituted in the container. Volumes to be considered for reconstituting in the containers include, but are not limited to, about 0.1 ml, about 0.2 ml, about 0.3 ml, about 0.4 ml, about 0.5 ml, about 0.6 ml, about 0.7 ml, about 0.8 ml, about 0.9 ml, about 1.0 ml, about 1.1 ml, about 1.2 ml, about 1.3 ml, about 1.4 ml, about 1.5 ml, about 1.6 ml, about 1.7 ml, about 1.8 ml, about 1.9 ml, about 2.0 ml, about 2.1 ml, about 2.2 ml, about 2.3 ml, about 2.4 ml, about 2.5 ml, about 2.6 ml, about 2.7 ml, about 2.8 ml, about 2.9 ml, about 3.0 ml, about 3.1 ml, about 3.2 ml, about 3.3 ml, about 3.4 ml, about 3.5 ml, about 3.6 ml, about 3.7 ml, about 3.8 ml, about 3.9 ml, about 4.0 ml, about 4.1 ml, about 4.2 ml, about 4.3 ml, about 4.4 ml, about 4.5 ml, about 4.6 ml, about 4.7 ml, about 4.8 ml, about 4.9 ml, and about 5.0 ml.

In some embodiments, the container is an ampoule, vial, a needleless injection device or pre-filled syringe. Containers described herein may, in some instances, contain a UV-blocking agent which helps prevent degradation of components of the compositions.

The compositions can further comprise acceptable carriers that are physiologically acceptable to the administered patient and retain the therapeutic properties of the compounds with/in which it is administered. Acceptable carriers and their formulations are and generally described in, for example, Remington' pharmaceutical Sciences (18th Edition, ed. A. Gennaro, Mack Publishing Co., Easton, Pa. 1990). One exemplary carrier is physiological saline. The phrase “pharmaceutically acceptable carrier” as used herein means an acceptable material, composition or vehicle, such as a liquid or solid filler, diluent, excipient, and/or solvent involved in carrying or transporting the subject compounds from the administration site of one organ, or portion of the body, to another organ, or portion of the body. Each carrier is acceptable in the sense of being compatible with the other ingredients of the formulation and not injurious to a subject to whom it is administered. Nor should an acceptable carrier alter the specific activity of the subject compounds.

Compositions described herein may be used to treat one or more clinical manifestations and/or symptoms of aging related skin problems. The present inventors have identified new compositions that may be applied topically or injected into a patient to improve skin elasticity, skin luminosity, skin regeneration, skin hydration, smoothness and/or softness of skin (i.e., making the skin feel smoother and softer following treatment); the overall appearance of skin; evening out skin tone and texture; clarity and/or radiance of skin; making the skin look younger; and making wrinkles and fine lines appear softer and/or less prominent, etc.

In one embodiment, the reconstituted HA solutions provide HA in combination with a neurotoxin formulated for topical use or intradermal administration. For a composition comprising a neurotoxin to be effective, the compositions provided to a patient should contain suitable amounts of neurotoxin and other formulation ingredients required by the patient. In one embodiment, the neurotoxin is botulinum toxin or micro botulinum toxin.

The aqueous solution may optionally include an effective amount of an anesthetic such as lidocaine with epinephrine, which are used for providing patient comfort and reducing bruising. An aqueous solution may optionally include a preservative. Any number of preservatives may be used to increase shelf life of intramuscularly injectable formulations. Non-limiting examples of preservatives are, for example, benzyl alcohol, methylparaben, propylparaben, benzyl alcohol, thimerosal, m-Cresol and methyl p-hydroxybenzoate. In one embodiment, benzyl alcohol also acts as a mild anesthetic potential that may mitigate the pain of injection. In some embodiments, the composition does not include a preservative.

The aforementioned components of the bioactive formulations (compositions) can be combined in any ratio, and administered in any volume, as determined necessary.

### Microneedle Device

The compositions can be administered by any means and the administration is not limiting. In some embodiments, the compositions, methods and kits herein can employ a microneedle device to administer the compositions. A microneedle array can be used to deliver a drug directly to the dermis (the second layer of skin). In some embodiments, the microneedle arrays or needleless injector devices as disclosed herein deliver the bioactive agent or drug into the dermal and epidermal junction area. In another embodiment, the microneedle device does not penetrate into the dermal layer but only disrupts the superficial portion of the skin, referred to as stratum corneum.

In one embodiment, the invention provides a microneedle device comprising a reservoir component, wherein the reservoir component comprises
i) an effective amount of hyaluronic acid in solid form; and
ii) optionally an effective amount of one or more bioactive agents in solid form.
   In some embodiments, the one or more bioactive agents is elected from the group consisting of one or more vitamins, one or more minerals, retinol, retinoic acid, a bleaching/whitening agent, collagen, a neuromodulator, poly-L-lactic acid, an anesthetic and combinations thereof. In some embodiments, the hyaluronic acid and one or more bioactive agents are not in admixture. In some embodiments, the hyaluronic acid and one or more bioactive agents are in admixture.

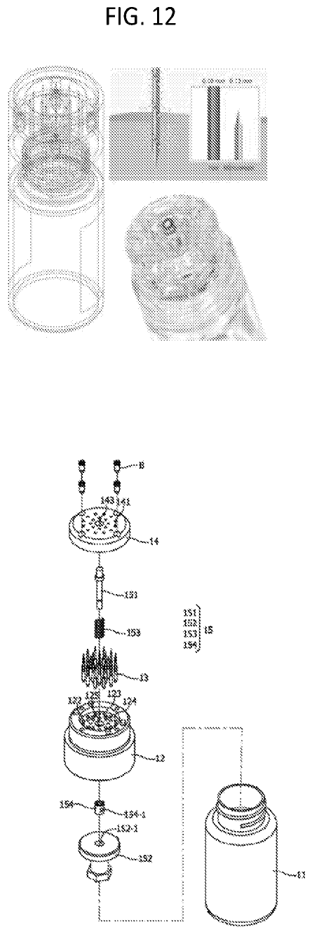

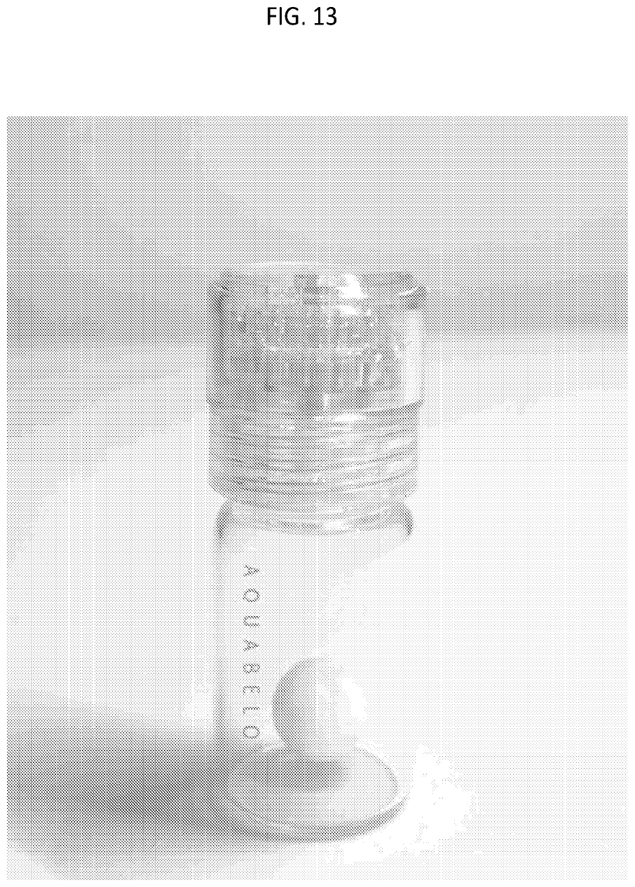

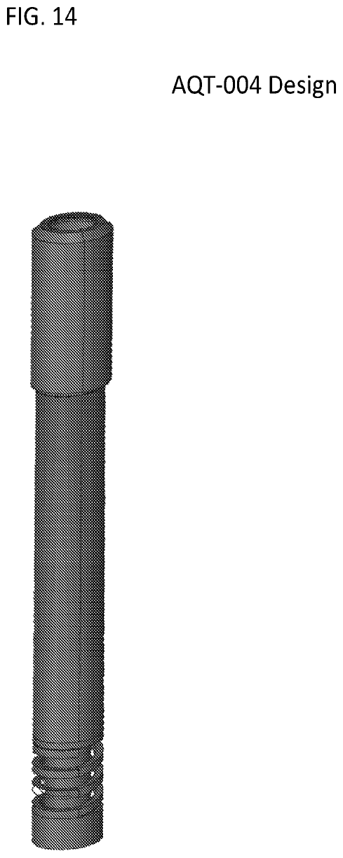

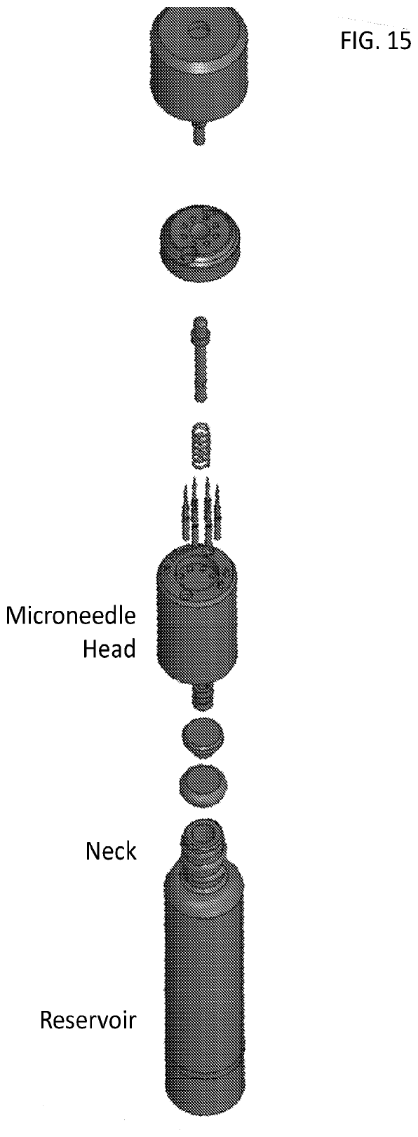

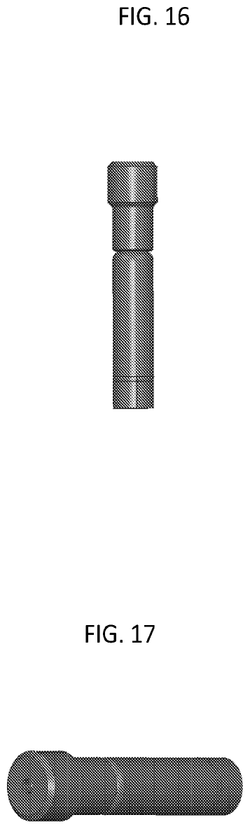

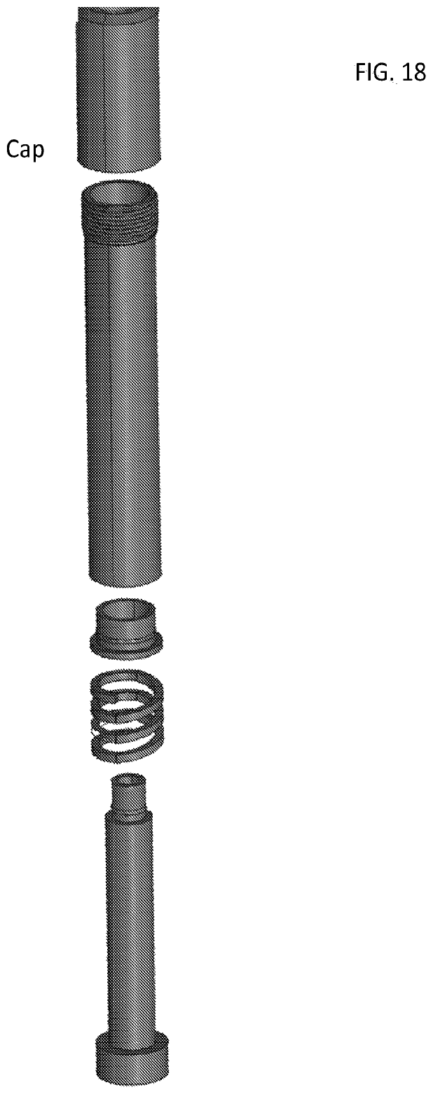

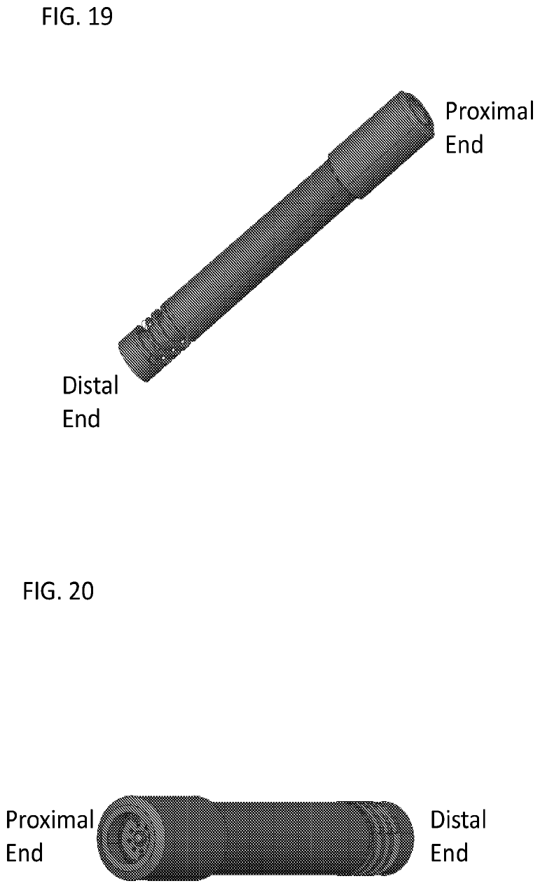

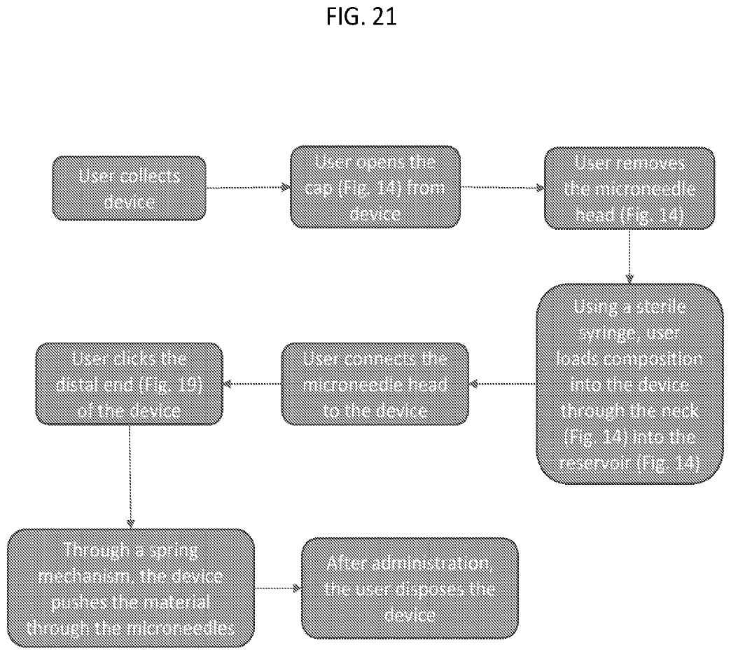

In some embodiments, the microneedle delivery device useful in the methods of the invention is depicted in FIG. 12. In some embodiments, the microneedle drug delivery device is as described in Korean Patent No. 10-1582822, which is incorporated by reference herein in its entirety. In some embodiments, the microneedle device useful in the methods of the invention is depicted in FIGS. 14-19.

In some embodiments, the microneedle delivery device comprises
i) one or more microneedles, wherein the microneedles are hollow or non-hollow, wherein one or multiple grooves are inset along an outer wall of the microneedles; and
ii) a reservoir that holds the composition to be delivered, wherein the reservoir is attached to or contains a means to encourage flow of the bioactive composition contained in the reservoir into the skin.

In some embodiments, the means to encourage flow of the composition contained in the reservoir into the skin is selected from the group consisting of a plunger, pump and suction mechanism. In some embodiments, the means to encourage flow of the composition contained in the reservoir into the skin is a mechanical spring loaded pump system.

In some embodiments, the microneedles have a single groove inset along the outer wall of the microneedle, wherein the single groove has a screw thread shape going clockwise or counterclockwise around the microneedle.

In some embodiments, the microneedles are from 0.1 mm to about 2.5 mm in length and from 0.01 mm to about 0.05 mm in diameter.

In some embodiments, the microneedles are made from a substance comprising gold.

In some embodiments, the plurality of microneedles comprises an array of microneedles in the shape of a circle.

In some embodiments, the microneedles are made of 24-carat gold plated stainless steel and comprise an array of about 10 to about 50 microneedles. In some embodiments, the array comprises 20 microneedles.

In some embodiments, the microneedle delivery device is repeatedly pressed against the subject's skin to deliver the composition to the area of the skin to be treated. In some embodiments, the microneedle delivery device is repeatedly pressed about 10, about 20, about 30, about 40, about 50, about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 or more times to administer the composition.

In some embodiments, the composition is administered by the microneedle delivery device with a repeated motion of penetrating the microneedle delivery device into the skin of the subject. In some embodiments, the composition is delivered into the skin by passing through the one or multiple grooves along the outer wall of the microneedle. In some embodiments, the microneedles are non-hollow.

In some embodiments, the administering comprises a repeated motion of penetrating the microneedle delivery device into the subject's skin in different areas of the subject's body.

In some embodiments, the microneedle delivery device comprises a single or an array of microneedles. In some embodiments, the microneedles will have one or multiple grooves inset along its outer wall. This structural feature of the dermal delivery device allows liquids stored in a reservoir at the base of each needle to travel along the needle shaft into the tissue.

In some embodiments, the microneedle array comprises from about 1 to about 500 microneedles, which will be anywhere from about 0.1 to about 2.5 mm in length and from 0.01 to about 0.5 mm in diameter, and be composed of any metal, metal alloy, metalloid, polymer, or combination thereof, such as gold, steel, silicon, PVP (polyvinylpyrrolidone), etc. The microneedles will each have one or more recesses running a certain depth into the outer wall to allow for flow of the substance to be delivered down the microneedle and into the dermis; these recesses can be in a plurality of shapes, including but not limited to: straight line, cross shape (+), flat shape (−), or screw thread shape going clockwise or counterclockwise. The array will be in any shape or combination of shapes, continuous, or discontinuous. The list of possible shapes includes, but is not limited to, circles, triangles, rectangles, squares, rhomboids, trapezoids, and any other regular or irregular polygons. The array can be attached to a reservoir to hold the substances to be delivered, and this reservoir will be any volume (0.25 mL to 5 mL), shape, color, or material (glass, metal, alloy, or polymer), as determined necessary. This reservoir will itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

The microneedle delivery device is capable of delivering compositions directly to the epidermal, dermal and subcuticular layers of the skin. Therefore, it should be understood that further embodiments developed for use with non-hollow or hollow microneedle systems of delivery by those skilled in the art fall within the spirit and scope of this disclosure.

In another aspect, a microneedle device for use in the methods described herein is a device such as described in U.S. Pat. No. 8,257,324, which is hereby incorporated by reference. Briefly, the devices include a substrate to which a plurality of hollow microneedles are attached or integrated, and at least one reservoir, containing a bioactive formulation, selectably in communication with the microneedles, wherein the volume or amount of composition to be delivered can be selectively altered. The reservoir can be, for example, formed of a deformable, preferably elastic, material. The device typically includes a means, such as a plunger, for compressing the reservoir to drive the bioactive formulation from the reservoir through the microneedles, A reservoir, can be, for example, a syringe or pump connected to the substrate. A device, in some instances, comprises: a plurality of hollow microneedles (each having a base end and a tip), with at least one hollow pathway disposed at or between the base end and the tip, wherein the microneedles comprise a metal; a substrate to which the base ends of the microneedles are attached or integrated; at least one reservoir in which the material is disposed and which is in connection with the base end of at least one of the microneedles, either integrally or separably; a sealing mechanism interposed between the at least one reservoir and the substrate, wherein the sealing mechanism comprises a fracturable barrier; and a device that expels the material in the reservoir into the base end of at least one of the microneedles and into the skin. The reservoir comprises a syringe secured to the substrate, and the device that expels the material comprises a plunger connected to a top surface of the reservoir. The substrate may be adapted to removably connect to a standard or Luer-lock syringe. In one instance, the device may further include a spring engaged with the plunger. In another instance, the device may further include an attachment mechanism that secures the syringe to the device. In another instance, the device may further include a sealing mechanism that is secured to the tips of the microneedles. In another instance, the device may further include means for providing feedback to indicate that delivery of the material from the reservoir has been initiated or completed. An osmotic pump may be included to expel the material from the reservoir. One or more microneedles may be disposed at an angle other than perpendicular to the substrate. In certain instances, the at least one reservoir comprises multiple reservoirs that can be connected to or are in communication with each other. The multiple reservoirs may comprise a first reservoir and a second reservoir, wherein the first reservoir contains a solid formulation and the second reservoir contains a liquid carrier for the solid formulation. A fracturable barrier for use in the devices can be, for example, a thin foil, a polymer, a laminate film, or a biodegradable polymer. The device may further comprise, in some instances, means for providing feedback to indicate that the microneedles have penetrated the skin.

In some embodiments, the device can include, in some instances, a single or plurality of solid, screw-type microneedles, of single or varied length. Typically the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrolidone) etc. The screw-shape dimensions may be variable. For example, in one embodiment the screw-shape may be a tight coiled screw shape, whereas in another embodiment the screw-shape might be a loose coiled screw shape whereby the screw threads in one embodiment lie closely together along the outer edge of the needle and, in another embodiment, the screw threads lie far from each other along the outer edge of the needle.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

A device described herein may contain, in certain instances, about twenty screw thread design surgical grade microneedles. Each microneedle has a diameter that is thinner than a human hair and may be used for direct dermal application. In one instance, a microneedle has a diameter of less than about 0.18 mm. In another instance, a microneedle has a diameter of about 0.15 mm, about 0.14 mm, about 0.13 mm, about 0.12 mm, about 0.11 mm, or about 0.10 mm. Each microneedle may be plated with 24 carat gold. The device allows for targeted and uniform delivery of a composition comprising the composition into the skin in a process that is painless compared to injectables. Administration can result in easy and precise delivery of a composition comprising the composition with generally no bruising, pain, swelling and bleeding caused by the injection.

The device may include means, manual or mechanical, for compressing the reservoir, creating a vacuum, or otherwise using gravity or pressure to drive the composition from the reservoir through the microneedles or down along the sides of the microneedle. The means can include a plunger, pump or suction mechanism. In another embodiment, the reservoir further includes a means for controlling rate and precise quantity of drug delivered by utilizing a semi-permeable membrane, to regulate the rate or extent of drug which flows along the shaft of the microneedles. The microneedle device enhances transportation of drugs across or into the tissue at a useful rate. For example, the microneedle device must be capable of delivering drug at a rate sufficient to be therapeutically useful. The rate of delivery of the drug composition can be controlled by altering one or more of several design variables. For example, the amount of material flowing through the needles can be controlled by manipulating the effective hydrodynamic conductivity (the volumetric through-capacity) of a single device array, for example, by using more or fewer microneedles, by increasing or decreasing the number or diameter of the bores in the microneedles, or by filling at least some of the microneedle bores with a diffusion-limiting material. It can be preferred, however, to simplify the manufacturing process by limiting the needle design to two or three “sizes” of microneedle arrays to accommodate, for example small, medium, and large volumetric flows, for which the delivery rate is controlled by other means.

Other means for controlling the rate of delivery include varying the driving force applied to the drug composition in the reservoir. For example, in passive diffusion systems, the concentration of drug in the reservoir can be increased to increase the rate of mass transfer. In active systems, for example, the pressure applied to the reservoir can be varied, such as by varying the spring constant or number of springs or elastic bands. In either active or passive systems, the barrier material can be selected to provide a particular rate of diffusion for the drug molecules being delivered through the barrier at the needle inlet.

The array may be in any shape or combination of shapes, continuous, or discontinuous. The list of possible shapes includes, but is not limited to, circles, triangles, rectangles, squares, rhomboids, trapezoids, and any other regular or irregular polygons.

The array may be attached to a reservoir to hold the substances to be delivered, and this reservoir may be any volume (about 0.25 mL to about 5 mL), shape, color, or material (glass, metal, alloy, or polymer), as determined necessary.

This reservoir can itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

In some embodiments, the device can include a single or plurality of solid, screw-type microneedles, of single or varied lengths housed in a plastic or polymer composite head which embodies a corrugated rubber connector. In some embodiments, the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrolidone) etc. The screw-shape dimensions may be variable. For example, in one embodiment the screw-shape may be a tight coiled screw shape, whereas in another embodiment the screw-shape might be a loose coiled screw shape. The corrugated rubber connector is a unique advantage conferring feature which bestows the microneedle head with a universally adoptable feature for interfacing the micro needle cartridges with multiple glass and or plastic vials, reservoirs and containers as well as electronic appendages for an altogether enhanced adjunct liquid handling, security and surveillance utility.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

In one embodiment, the direct application device comprises a single or an array of microneedles that will serve not only as an anchor to the skin, but also as a collagen stimulator platform (via collagen induction therapy) to accelerate skin healing or combat age-related decreases in collagen neosynthesis. Each microneedle will have one or multiple grooves inset along its outer wall. This structural feature of the dermal delivery device allows liquids stored in a reservoir at the base of each needle to travel along the needle shaft into the tissue. Altogether, this innovation is a functional treatment regimen applicator that enables the optimal restorative efficacy of bioactive formulations delivered beneath the surface of the skin.

A microneedle array can consist of from about 1 to about 500 microneedles, which will be anywhere from about 0.1 to about 2.5 mm in length and from 0.01 to about 0.5 mm in diameter, and be composed of any metal, metal alloy, metalloid, polymer, or combination thereof, such as gold, steel, silicon, PVP (polyvinylpyrrolidone), etc. The microneedles can each have one or more recesses running a certain depth into the outer wall to allow for flow of the substance to be delivered down the microneedle and into the dermis; these recesses can be in a plurality of shapes, including but not limited to: straight line, cross shape (+), flat shape (−), or screw thread shape going clockwise or counterclockwise. The array will be in any shape or combination of shapes, continuous, or discontinuous. The list of possible shapes includes, but is not limited to, circles, triangles, rectangles, squares, rhomboids, trapezoids, and any other regular or irregular polygons. The array will be attached to a reservoir to hold the substances to be delivered, and this reservoir will be any volume (0.25 mL to 5 mL), shape, color, or material (glass, metal, alloy, or polymer), as determined necessary. This reservoir will itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

The delivered substances may be of varying viscosities and concentration, from 0.01% to 100%, and will be administered via the microneedle array either independently or in conjunction with the aforementioned compositions.

The reservoir will itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

A cadre of microneedles housed in a plastic or polymer composite head can be used to deliver treatment solutions, directly to the dermis, the second layer of skin or the topical layer of skin. The application of a mechanical load to the pin of the spring lock system enables the micro needles to puncture the epidermal barrier and deliver the desired substances directly to the dermis for faster, more efficient, and more effective absorption by the skin. The Spring Plate mechanism, housed in the plastic or polymer composite cartridge, is effectively the interface whereby the manual direct application mechanism calibrates the controlled delivery of the treatment solution into the skin.

Provided herein is a system, comprising a bioactive formation and a microneedle delivery system, wherein the bioactive formulation comprises hyaluronic acid HA crystals and adjuvant materials; a neurotoxin, hyaluronic acid and a pharmaceutically acceptable excipient.

Other suitable microneedle devices such as pens, rollers, and patches, may be used to delivery or collect liquids when interfaced with tissue. They can be manually or electronically applied to achieve a stamping motion, or rolled over the skin. Such devices also include, in some instances, an autoinjector.

Provided herein is a system, comprising a bioactive formation and a microneedle delivery system, wherein the bioactive formulation comprises: HA, neurotoxin, one or more vitamins, one or more minerals, one or more retinols, one or more antioxidants, one or more growth factors, one or more bleaching/whitening agents, or a combination thereof.

In another embodiment, provided herein is a system comprising a microneedle delivery device and a bioactive formulation, comprising (a) a single or an array of microneedles with a channel for liquid form to pass through; (b) a reservoir chamber with bioactive compounds or formulations customized or prefilled; (c) a plunger that releases the said compounds or formulations; (d) an optional security material to anti-reverse lock the microneedles and the chamber; (e) an optional adapter to fit any reservoir chamber; (f) an optional RFID smart label connecting to artificial intelligence portal; (g) an optional cloud-based informatics platform connected to system (big data predictive analytics); (h) an optional UV blocking agent for the chamber; and (i) an optional addition of a patch form using the groove in the microneedles for the purpose of sustained and/or extended release of the compositions, formulations or microchips.

The optional security material may comprise a Butyl Rubber O-Ring for optimizing the end-user utility and or experience. In one embodiment, the optimized butyl rubber O-ring confers pivotal advantages for the delivery of therapeutic treatment solutions to a tissue bed. In another embodiment, the optimized butyl rubber O-ring enable the overall improvement in performance and or utility of the underlying medical device. In another embodiment, the optimized butyl rubber O-ring is a flat or circular Butyl Rubber O-Ring. In another embodiment, the optimized butyl rubber O-ring serves as a liquid handling and leakage prevention seal, and improves the system efficiency characterized by an airtight mechanism for applying uniform volumes of treatment solutions. In another embodiment, the optimized butyl rubber O-ring further substantiates the overall suitability of the material in the context of medical device performance.

### Kits

In another aspect, the invention provides a kit for use in the methods herein, comprising the compositions or microneedle devices as described herein, optionally comprising instructions for use and optionally further comprising one or more aqueous solvents for dissolving the hyaluronic acid and/or one or more bioactive agents. In some embodiments, the solvent is a buffered solution. In some embodiments, the solution is saline solution. In some embodiments, the solvent is water. In some embodiments, the kit comprises one or more anesthetics. In some embodiments, the kit further comprises one or more containers or packets comprising one or more bioactive agents in solid or liquid form. In some embodiments, the one or more bioactive agents is selected from the group consisting of one or more vitamins, one or more minerals, retinol, retinoic acid, a bleaching/whitening agent, collagen, a neuromodulator, poly-L-lactic acid, an anesthetic and combinations thereof.

What is claimed is:

1. A method of administering a composition comprising hyaluronic acid to a subject's skin, comprising
i) providing an effective amount of hyaluronic acid in solid form;
ii) reconstituting the hyaluronic acid in an aqueous solution; and
iii) administering the solution comprising hyaluronic acid to the subject's skin.

2. The method of claim 1, wherein hyaluronic acid in solid form is crystalline.

3. The method of any of claim 1 or 2, wherein the hyaluronic acid in solid form is freeze dried.

4. The method of any of claim 1 or 2, wherein the hyaluronic acid in solid form is vacuum dried or spray dried.

5. The method of any of claim 1 or 2, wherein the hyaluronic acid in solid form is oven dried.

6. The method of any of claims 1-5, wherein the hyaluronic acid is crosslinked.

7. The method of any of claims 1-5, wherein the hyaluronic acid is non-crosslinked.

8. The method of any of claims 1-5, wherein at least a portion of the hyaluronic acid is crosslinked.

9. The method of any of claims 1-8, wherein the hyaluronic acid in solid form is substantially pure.

10. The method of any of claims 1-8, wherein the hyaluronic acid in solid form is in admixture with an effective amount of one or more additional bioactive agents.

11. The method of claim 10, wherein the one or more bioactive agents in admixture is freeze dried, vacuum dried and/or oven dried.

12. The method of any of claims 1-9, wherein the hyaluronic acid is reconstituted in a solution comprising an effective amount of one or more bioactive agents.

13. The method of any of claims 1-9, wherein the reconstituted solution of hyaluronic acid is combined with an effective amount of one or more bioactive agents in solid and/or liquid form.

14. The method of claim 13, wherein the one or more bioactive agents in solid form is freeze dried, vacuum dried, spray dried and/or oven dried.

15. The method of claim 13, wherein the one or more bioactive agents in liquid form comprises an aqueous solution.

16. The method of any of claims 9-15, wherein the one or more bioactive agents comprises a neuromodulator, vitamin, mineral, PLLA (poly-L-lactic acid) or any combination thereof.

17. The method of claim 16, wherein the neuromodulator comprises botulinum toxin (e.g., botulinum toxin of serotype A, B, C, D, E, F or G).

18. The method of any of claims 1-17, wherein the hyaluronic acid in solid form or admixture is in the shape of flakes, a ball, a cube, or a star.

19. The method of any of claims 1-18, wherein the solution is administered to the subject's skin using a microneedle delivery device.

20. The method of claim 19, wherein the microneedle delivery device comprises
i) a plurality of microneedles, wherein the microneedles are hollow or non-hollow, wherein one or multiple grooves are inset along an outer wall of the microneedles; and
ii) a reservoir capable of holding the solution to be delivered to a subject's skin,
wherein the reservoir is attached to or contains a means to encourage flow of the solution into the tissue; wherein the solution is capable of being delivered into the skin by passing through the one or multiple grooves along the outer wall of the microneedle by a repeated motion of penetrating the microneedle delivery device into the skin of the subject.

21. The method of any of claims 1-20, wherein the hyaluronic acid in solid form is provided in a reservoir component of a microneedle device.

22. The method of claim 21, wherein the hyaluronic acid is reconstituted in the reservoir component.

23. The method of claim 22, wherein the reservoir component is made of glass.

24. The method of any of claims 19-23, wherein the microneedles are non-hollow.

25. The method of any of claims 20-24, wherein the means to encourage flow of the solution into the tissue is selected from the group consisting of a plunger, pump and suction mechanism.

26. The method of any of claims 20-24, wherein the means to encourage flow of the solution into the tissue is a mechanical spring loaded pump system.

27. The method of any of claims 19-26, wherein the microneedles have a single groove inset along the outer wall of the microneedle, wherein the single groove has a screw thread shape going clockwise or counterclockwise around the microneedle.

28. The method of any of claims 19-27, wherein the microneedles are from 0.1 mm to about 1.0 mm in length and from 0.01 mm to about 0.2 mm in diameter.

29. The method of any of claims 19-28, wherein the microneedles are composed of gold.

30. The method of any of claims 20-29, wherein the plurality of microneedles comprises an array of microneedles in the shape of a circle.

31. The method of any of claims 20-30, wherein the microneedles are made of 24-carat gold plated stainless steel and comprise an array of 20 microneedles.

32. The method of any of claims 10-31, wherein the one or more bioactive agents comprises a vitamin, a mineral, retinol, retinoic acid, a bleaching/whitening agent, stem cells, collagen, a neurotoxin, platelet-rich plasma, poly-L-lactic acid, an anesthetic or combinations thereof.

33. The method of any of claims 1-32, wherein the solution is substantially free of preservatives.

34. The method of any of claims 1-33, wherein the solution has a viscosity that enables efficient injection with a microneedle device.

35. A solid composition comprising an effective amount of hyaluronic acid in admixture with an effective amount of one or more bioactive agents.

36. The composition of claim 35, wherein the one or more bioactive agents is elected from the group consisting of one or more vitamins, one or more minerals, retinol, retinoic acid, a bleaching/whitening agent, collagen, a neuromodulator, poly-L-lactic acid, an anesthetic and combinations thereof.

37. The composition of any of claim 35 or 36, wherein the hyaluronic acid and/or bioactive agent is freeze dried.

38. The composition of any of claim 35 or 36, wherein the hyaluronic acid and/or bioactive agent is vacuum dried or spray dried.

39. The composition of any of claim 35 or 36, wherein the hyaluronic acid and/or bioactive agent is oven dried.

40. The composition of any of claims 35-39, wherein the hyaluronic acid is crosslinked.

41. The composition of any of claims 35-39, wherein the hyaluronic acid is non-crosslinked.

42. The composition of any of claims 35-39, wherein at least a portion of the hyaluronic acid is crosslinked.

43. The composition of any of claims 35-42, wherein the hyaluronic acid in solid form is substantially pure.

44. The composition of any of claims 35-42, wherein the one or more bioactive agents comprises a neuromodulator.

45. The composition of claim 44, wherein the neuromodulator comprises botulinum toxin (e.g., botulinum toxin of serotype A, B, C, D, E, F or G).

46. The composition of any of claims 35-45, wherein the admixture is in the shape of flakes, a ball, a cube, or a star.

47. The composition of any of claims 35-46, wherein the composition is provided in a container.

48. The composition of claim 47, wherein the container is a reservoir component of a microneedle delivery device.

49. The composition of claim 48, wherein the microneedle delivery device comprises:
i) a plurality of microneedles, wherein the microneedles are hollow or non-hollow, wherein one or multiple grooves are inset along an outer wall of the microneedles; and
ii) a reservoir component capable of holding the solid composition,
wherein the reservoir is attached to or contains a means to encourage flow of a solution into skin; wherein the solution is capable of being delivered into the skin by passing through the one or multiple grooves along the outer wall of the microneedle by a repeated motion of penetrating the microneedle delivery device into the skin of a subject.

50. The composition of claim 47-49, wherein the reservoir component is made of glass.

51. The composition of any of claims 48-50, wherein the microneedles are non-hollow.

52. The composition of any of claims 49-51, wherein the means to encourage flow of the solution into the tissue is selected from the group consisting of a plunger, pump and suction mechanism.

53. The composition of any of claims 49-51, wherein the means to encourage flow of the solution into the tissue is a mechanical spring loaded pump system.

54. The composition of any of claims 48-53, wherein the microneedles have a single groove inset along the outer wall of the microneedle, wherein the single groove has a screw thread shape going clockwise or counterclockwise around the microneedle.

55. The composition of any of claims 48-54, wherein the microneedles are from 0.1 mm to about 1.0 mm in length and from 0.01 mm to about 0.2 mm in diameter.

56. The composition of any of claims 48-55, wherein the microneedles are composed of gold.

57. The composition of any of claims 49-56, wherein the plurality of microneedles comprises an array of microneedles in the shape of a circle.

58. The composition of any of claims 48-57, wherein the microneedles are made of 24-carat gold plated stainless steel and comprise an array of 20 microneedles.

59. The composition of any of claims 35-58, wherein the admixture is substantially free of preservatives.

60. An injectable solution comprising the composition of any of claims 35-59, wherein the composition has been reconstituted in aqueous solution, wherein the solution has a viscosity that enables efficient injection with a microneedle device.

61. A microneedle device comprising a reservoir component, wherein the reservoir component comprises
iii) an effective amount of hyaluronic acid in solid form; and
iv) optionally an effective amount of one or more bioactive agents in solid form.

62. The microneedle device of claim 61, wherein the device comprises hyaluronic acid and the one or more bioactive agents.

63. The microneedle device of any of claim 61 or 62, wherein the hyaluronic acid and one or more bioactive agents are not in admixture.

64. The microneedle device of any of claim 61 or 62, wherein the hyaluronic acid and one or more bioactive agents are in admixture.

65. The microneedle device of any of claims 61-64, wherein the one or more bioactive agents is selected from the group consisting of one or more vitamins, one or more minerals, retinol, retinoic acid, a bleaching/whitening agent, collagen, a neuromodulator, poly-L-lactic acid, an anesthetic and combinations thereof.

66. The microneedle device of any of claims 61-65, wherein hyaluronic acid and/or bioactive agent is crystalline.

67. The microneedle device of any of claims 61-66, wherein the hyaluronic acid and/or bioactive agent is freeze dried.

68. The microneedle device of any of claims 61-66, wherein the hyaluronic acid and/or bioactive agent is vacuum dried.

69. The microneedle device of any of claims 61-66, wherein the hyaluronic acid and/or bioactive agent is oven dried.

70. The microneedle device of any of claims 61-69, wherein the hyaluronic acid is crosslinked.

71. The microneedle device of any of claims 61-69, wherein the hyaluronic acid is non-crosslinked.

72. The microneedle device of any of claims 61-69, wherein at least a portion of the hyaluronic acid is crosslinked.

73. The microneedle device of any of claims 61-72, wherein the hyaluronic acid in solid form is substantially pure.

74. The microneedle device of any of claims 61-72, wherein the one or more bioactive agents comprises a neuromodulator.

75. The microneedle device of claim 74, wherein the neuromodulator comprises botulinum toxin (e.g., botulinum toxin of serotype A, B, C, D, E, F or G).

76. The microneedle device of any of claims 61-75, wherein the hyaluronic acid and/or one or more bioactive agents or admixture thereof is in the shape of flakes, a ball, a cube, or a star.

77. The microneedle device of any of claims 61-75, wherein the microneedle delivery device comprises:
i) a plurality of microneedles, wherein the microneedles are hollow or non-hollow, wherein one or multiple grooves are inset along an outer wall of the microneedles; and
ii) the reservoir component,
wherein the reservoir is attached to or contains a means to encourage flow of a solution comprising bioactive agents into skin; wherein the solution is capable of being delivered into the skin by passing through the one or multiple grooves along the outer wall of the microneedle by a repeated motion of penetrating the microneedle delivery device into the skin of a subject.

78. The microneedle device of any of claims 61-77, wherein the reservoir component is made of glass.

79. The microneedle device of any of claims 61-78, wherein the microneedles are non-hollow.

80. The microneedle device of any of claims 77-79, wherein the means to encourage flow of the solution into the tissue is selected from the group consisting of a plunger, pump and suction mechanism.

81. The microneedle device of any of claims 77-79, wherein the means to encourage flow of the solution into the tissue is a mechanical spring loaded pump system.

82. The microneedle device of any of claims 61-81, wherein the microneedles have a single groove inset along the outer wall of the microneedle, wherein the single groove has a screw thread shape going clockwise or counterclockwise around the microneedle.

83. The microneedle device of any of claims 61-82, wherein the microneedles are from 0.1 mm to about 1.0 mm in length and from 0.01 mm to about 0.2 mm in diameter.

84. The microneedle device of any of claims 61-83, wherein the microneedles are composed of gold.

85. The microneedle device of any of claims 77-84, wherein the plurality of microneedles comprises an array of microneedles in the shape of a circle.

86. The microneedle device of any of claims 61-85, wherein the microneedles are made of 24-carat gold plated stainless steel and comprise an array of 20 microneedles.

87. The microneedle device of any of claims 61-86, wherein the hyaluronic acid and/or one or more bioactive agents are substantially free of preservatives.

88. A kit comprising the microneedle device of any of claims 61-86 optional comprising instructions for use and optionally further comprising one or more aqueous solutions for dissolving the hyaluronic acid and/or one or more bioactive agents.

89. The kit of claim 88, wherein the solution is saline solution.

90. The kit of claim 88, wherein the solution is a buffered solution.

91. The kit of any of claims 88-90, further comprising one or more anesthetics.

92. The kit of any of claims 88-91, further comprising a container comprising one or more bioactive agents in solid or liquid form.

93. The kit of claim 92, wherein the one or more bioactive agents is selected from the group consisting of one or more vitamins, one or more minerals, retinol, retinoic acid, a bleaching/whitening agent, collagen, a neuromodulator, poly-L-lactic acid, an anesthetic and combinations thereof.
